(19) United States
(12) Patent Application Publication
(10) Pub. No.: US 2024/0299557 A1
(43) Pub. Date: Sep. 12, 2024

(54) COMPOUND FOR DEGRADING DEOXYRIBONUCLEIC ACID (DNA) POLYMERASE, AND USE THEREOF

(71) Applicant: TAIBIDI PHARMACEUTICAL TECHNOLOGY (SHIJIAZHUANG) CO., LTD., Shijiazhuang, Hebei (CN)

(72) Inventors: Xiangdong SU, Shijiazhuang (CN); Fengmin LU, Shijiazhuang (CN); Fei QI, Shijiazhuang (CN); Tianle WEN, Shijiazhuang (CN); Mingjie BAI, Shijiazhuang (CN); Jinxu WANG, Shijiazhuang (CN)

(73) Assignee: TAIBIDI PHARMACEUTICAL TECHNOLOGY (SHIJIAZHUANG) CO., LTD., Shijiazhuang, Hebei (CN)

(21) Appl. No.: 18/273,212

(22) PCT Filed: Jan. 21, 2022
(86) PCT No.: PCT/CN2022/073159

§ 371 Date: Jul. 19, 2023

(30) Foreign Application Priority Data
Jan. 22, 2021 (CN) 202110094088.8
Jan. 20, 2022 (CN) 202210064310.4

(51) Int. Cl. A61K 47/55; A61K 47/54; A61K 47/60; A61P 31/20; G01N 33/569

(52) U.S. Cl. CPC A61K 47/55; A61K 47/545; A61K 47/548; A61K 47/549; A61K 47/60; A61P 31/20; G01N 33/56983; C12N 2503/00; G01N 2333/91245; G01N 2469/10

(57) ABSTRACT

The present invention relates to the field of biomedicine, and specifically relates to a proteolysis-targeting chimera (PROTAC) compound. The structure of the PROTAC compound can be represented by general formula LGP-LK-LGE, wherein LGP is a ligand for binding a deoxyribonucleic acid (DNA) polymerase; LGE is a ligand for binding an E3 ubiquitin ligase; and LK is a linker linking the two above ligands. The compound prevents virus replication and kills viruses by means of degrading a deoxyribonucleic acid (DNA) polymerase that inhibits the viruses, and thus performs the function of treating and intervening in viral infectious diseases such as hepatitis B and secondary diseases, and the acquired immunodeficiency syndrome.

### TECHNICAL FIELD

The present disclosure relates to the field of biomedicine, in particular to a class of proteolysis-targeting chimera (PROTAC) compounds, which has the formula of LGP-LK-LGE, wherein LGP is a ligand binding a deoxyribonucleic acid (DNA) polymerase, LGE is a ligand of E3 ubiquitin ligase, and LK is a linker linking two ligands. The disclosed compounds degrade deoxyribonucleic acid (DNA) polymerase, leading to the inhibition of viral replication, and cause viruses death. Therefore, the disclosed compounds play a therapeutic role in the treatment and intervention of viral infectious diseases including hepatitis B, its complications and acquired immune deficiency syndrome.

### BACKGROUND

Many pathological processes can be attributed to uncontrolled deoxyribonucleic acid (DNA) replication, including viral or bacterial infections, autoimmune diseases, tumors, etc. During DNA replication, DNA polymerase catalyzes the polymerization of nucleotide monomers using DNA or RNA as a template to form long-chain polymer nucleic acids. The regulation of DNA polymerase is an important means to influence and interfere with DNA replication. As highly prevalent diseases, viral infections such as hepatitis, acquired immunodeficiency syndrome (AIDS), severe acute respiratory syndrome (SARS) can be characterized by extensive viral DNA synthesis. As a key enzyme in DNA synthesis, viral endogenous polymerase is a vital component for maintaining life cycle of various viruses, including hepatitis viruses, human immunodeficiency viruses and influenza viruses. The viral endogenous DNA polymerase is a critical target for the development of antiviral drugs.

Hepatitis B virus (HBV) is a DNA virus from the Hepadnaviridae family. HBV cause acute and chronic hepatitis, and the HBV infection is the major cause of liver cancer and liver cirrhosis. World Health Organization reported that about 2 billion people in the world have been infected with hepatitis B virus, of which more than 350 million people are chronically infected, and about 786,000 people die every year from liver failure, liver cirrhosis and primary hepatocellular carcinoma (HCC) caused by HBV infection. The molecular biology study of HBV properties provided support to search for therapeutics based on novel mechanisms of action. The HBV genome is partially double-stranded circular DNA, with the minus strand being about 3.2 kb, and the plus strand being about 50-100% of the minus strand. Its genome contains four open reading frames (ORF), corresponding to the genes encoding polymerase (P protein), nuclear protein (C protein), surface protein (S protein) and X protein. Among the proteins expressed by these HBV genes, polymerase, surface protein and nucleoprotein are structural proteins, while X protein has regulatory functions. The HBV polymerase gene accounts for 80% of the entire viral genome and overlaps with the coding regions of other genes. Based on different genotype, it can encode a P protein consisting of 832 to 845 amino acids, which is the DNA polymerase (Seeger C, Mason W S. Hepatitis B virus biology. Microbiol Mol Biol Rev. 2000; 64 (1):51-68). This gene plays multiple roles in viral genome replication. In addition to the function of nucleic acid replication, the expressed DNA polymerase (P protein) is also served as a necessary structural component along with capsid protein, pre-genomic RNA and cytokines to participate in virus packaging to form immature virus particles. HBV DNA polymerase acts in hepatocytes to synthesize DNA and attach to short chains to form a complete double helix of the HBV genome, and further generates pregenomic mRNA and mRNA of nucleoprotein, surface protein and regulatory protein (X protein) through the action of RNA polymerase The latter acts as a messenger and participates in the translation and synthesis of viral proteins.

Analysis of the amino acid sequence encoding the open reading frame P region of the HBV genome reveals that portion of the region is highly conserved, similar to the RNA-dependent DNA polymerase region and the ribonuclease H (RNase H) region of retroviruses (Bartenschlager R, Schaller H. EMBO J. 1992 September; 11(9):3413-20). Studies have shown that HBV DNA polymerase constitute four domains starting from the 5′ end: the terminal protein domain (TP domain), spacer domain (spacer), reverse transcriptase domain (RT domain) and ribonuclease domain (RNaseH domain). These different domains are associated with various steps in the viral replication process. The terminal proteins have multiple functions, mainly as primers when pregenomic RNA reverse-transcribes minus strand DNA. Terminal proteins also inhibit the activation of interferon-inducible genes in host cells and interfere with the effect of interferon. DNA polymerases play a crucial role in the production of viral genomes, forming structures called replisomes with nucleoproteins and pregenomic mRNAs. When the replisome is formed, the minus strand DNA is synthesized by the reverse transcription action of HBV polymerase, while the plus strand DNA is made by the action of DNA-dependent DNA polymerase, which in turn produces pregenomic mRNA. Before DNA plus strand synthesis, the activity of ribonuclease H is required to remove RNA in the RNA-DNA hybrid, leaving single-stranded DNA; the small fragments of RNA formed by degradation serve as primers for plus strand DNA synthesis. The amino acid homology analysis of the reverse transcription region of HBV DNA polymerase found that it contains five main functional regions, which are A region, B region, C region, D region and E region. The A, C and D regions are the binding regions of enzymes and nucleoside triphosphates, while the B and E regions are the regions for RNA template and primer positioning. The catalytic domain of DNA polymerase is located in the YMDD structure of the C region. These five regions contain highly conserved amino acid sequences, which are necessary to maintain reverse transcription activity.

Since the HBV DNA polymerase plays a crucial role in the reverse transcription and even the entire replication process of the virus, nucleoside compounds with the function of inhibiting reverse transcription of the polymerase are currently the primary drugs for the treatment of HBV infection. These drugs include nucleoside analogs such as Lamivudine, Telbivudine, Entecavir, Adefovir Dipivoxil, Tenofovir disoproxil and Tenofovir alafenamide fumarate. These nucleoside analogs are incorporated into the DNA chain of the polymerase, inhibit the reverse transcription activity of the HBV P protein, and irreversibly terminate the extension and synthesis of the new HBV DNA chain of the progeny virus, thereby preventing the virus from multiplying. However, nucleoside analogs had no significant effect on cccDNA and did not reduce pre-genomic RNA and mRNA, indicating that DNA-templated transcription and viral protein translation were not affected by the drug. Currently, the therapeutic methods targeting the reverse transcription function of polymerase can only inhibit the reproduction of the virus, but not eliminate HBV completely, achieving the purpose of curing hepatitis B. Furthermore, nucleoside drugs also develop drug resistance and rebound after drug withdrawal. Therefore, there is urgency to find anti-HBV drugs with new mechanisms of action. Since HBV DNA polymerase (P protein) has different functions in multiple stages of the virus life cycle, it is a promising development direction to design new types of drugs that can act on the whole P protein of HBV to block multiple functions.

There are two major protein degradation pathways in eukaryotic cells: autophagy and ubiquitin proteasome system. The ubiquitin-proteasome pathway is an efficient and specific protein degradation process that regulates the degradation of most proteins in cells. Protein degradation by ubiquitination plays an extremely important role in maintaining the levels of various proteins in cells, involving almost all life activities such as regulation of cell cycle, proliferation, apoptosis, metastasis, gene expression, and signal transmission. Ubiquitin is a highly conserved protein ubiquitous in eukaryotic cells, consisting of 76 amino acids. Ubiquitinated proteins can be transported to the 26S proteasome or entered into the lysosome for digestion and degradation. The ubiquitination of proteins is carried out through a series of enzymatic reactions. First, ubiquitin is linked to E1 through the formation of a high-energy thioester bond between the carboxyl group on its C-terminal glycine and the essential cysteine sulfhydryl group on ubiquitin activating enzyme E1 to become activated ubiquitin; secondly, the activated ubiquitin is transferred from ubiquitin activating enzyme E1 to ubiquitin conjugating enzyme E2; and finally, under the action of E3 ubiquitin ligase, ubiquitin molecules attached to ubiquitin conjugating enzyme E2 are attached to the substrate protein through covalent ligations of isomeric peptide bonds. The specificity of ubiquitin-mediated protein degradation depends on the specific recognition ability of substrate proteins by ubiquitin ligase E3.

Proteolytic targeting chimera (PROTAC) technology utilizes the intracellular ubiquitin-proteasome system to degrade specific target proteins. The technical feature is that the small molecule ligand that can bind to the target protein and the ligand of E3 ubiquitin ligase are connected through a linker fragment to form a bifunctional compound. By adjusting and optimizing the connection method, the size of the linker and other physical and chemical properties, the ligands at both ends of the PROTAC molecule are caused to bind to the target protein and E3 ubiquitin ligase, forming a target protein-PROTACs-E3 ligase ternary complex, thereby enabling target proteins are ubiquitinated and degraded by the proteasome system. PROTAC technology has the advantages that it can be used for the degradation of undruggable proteins, the degradation efficiency is strong, and the catalytic degradation can be maintained at low concentrations. At the same time, because the protein degradation mode of this technology is repeated iterations, it has better tolerance than traditional drugs in the case of target protein mutation. The technical difficulty of PROTAC is that the target protein ligand, the conformation and site of the E3 ubiquitin ligase ligand, the modification of the length and composition of the linker, and the concentration will all affect the formation and stability of the ternary complex, making it more challenging to control.

In summary, given the important role of these viral endogenous DNA polymerases in the process of viral replication, and the efficient and specific degradation of target proteins by the ubiquitin-proteasome system of host cells, the purpose of the present disclosure is to provide a class of proteolysis targeting chimeras (PROTAC). Such molecules can specifically degrade the viral endogenous DNA polymerase (P protein), thereby blocking the replication of the virus at multiple stages and achieving the purpose of treating viral infections.

### SUMMARY OF THE INVENTION

The present disclosure relates to the field of biomedicine, in particular to a class of compounds or pharmaceutically acceptable salts, solvates, hydrates, polymorphs, tautomers, geometric isomers, isotopic labels, metabolites or prodrugs thereof. Such compounds degrade deoxyribonucleic acid (DNA) polymerase which inhibit virus replication, kill virus. They play a key role in the treatment and intervention of viral infectious diseases. These compounds are proteolysis-targeting chimeras (PROTAC), whose structure can be represented by the general formula of LGP-LK-LGE, wherein LGP is a ligand binding a deoxyribonucleic acid (DNA) polymerase, LGE is a ligand of E3 ubiquitin ligase, and LK is a linker linking two ligands. That is a combination of molecular functional groups. The present disclosure provides a new class of deoxyribonucleic acid (DNA) polymerase degrading agents, which can effectively interfere with virus survival and replication, and treat diseases caused by virus infection, including hepatitis B, its complications and acquired immunodeficiency syndrome.

A class of proteolytic targeting chimeras with the general formula LGP-LK-LGE provided by the present disclosure can effectively degrade DNA polymerase (P protein) in HBV-infected cells, and can effectively inhibit HBV replication.

The present disclosure is achieved through the following aspects: A first aspect of the present disclosure provides a bifunctional compound, which is a proteolytic targeting chimera, and has the formula of LGP-LK-LGE, wherein LGP is a ligand binding a deoxyribonucleic acid (DNA) polymerase, LGE is a ligand of E3 ubiquitin ligase, and LK is a linker linking LGP and LGE.

A second aspect of the present disclosure relates to the compound provided above, wherein the bifunctional compound has the formula of LGP-LK-LGE also comprises a pharmaceutically acceptable salt, a solvate, a hydrate, a polymorph, a tautomer, a geometric isomer, an isotopic label, a metabolite or a prodrug thereof.

A third aspect of the present disclosure relates to the compound of LGP-LK-LGE provided above, wherein, the ligand binding a deoxyribonucleic acid (DNA) polymerase, LGP is selected from the group consisting of the following structures, or is selected from phosphorylated, bisphosphorylated or triphosphorylated product at any hydroxyl group of the following structures:

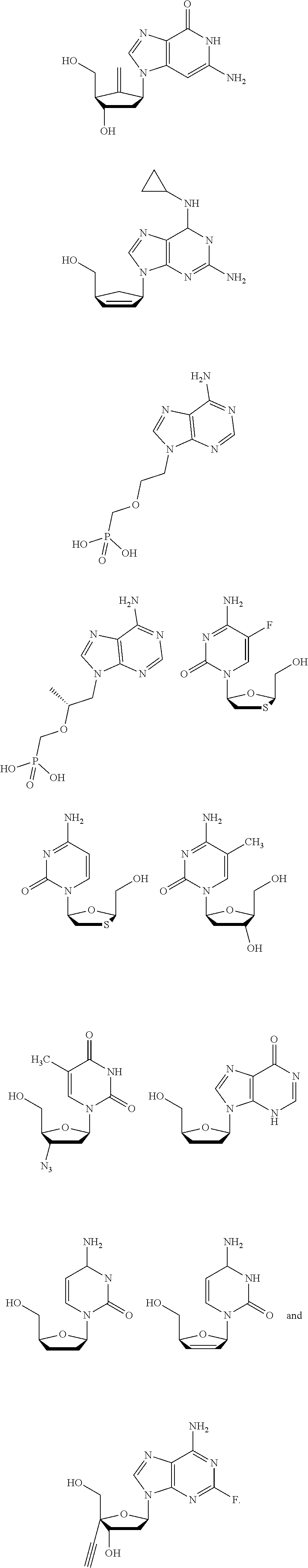

A fourth aspect of the present disclosure relates to the compound of LGP-LK-LGE provided above, wherein, the linker LK is linked to LGP through chemical bonds; wherein the LK is linked to the base of LGP or to the cyclopentose unit of LGP; further preferably, the linking position is shown in any of the following structures:

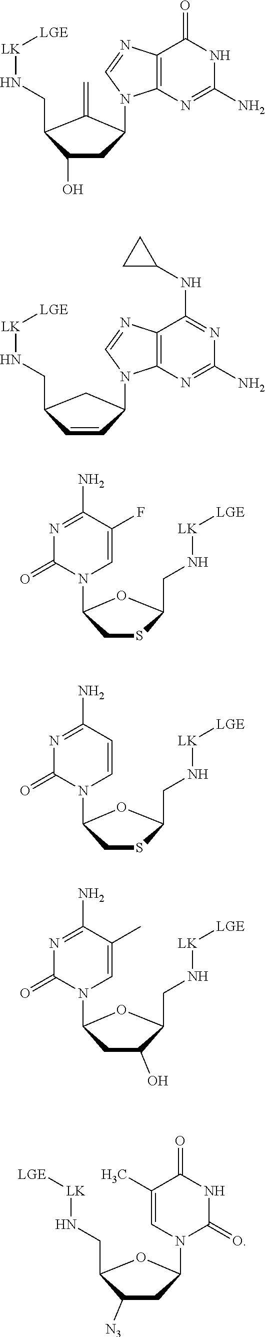

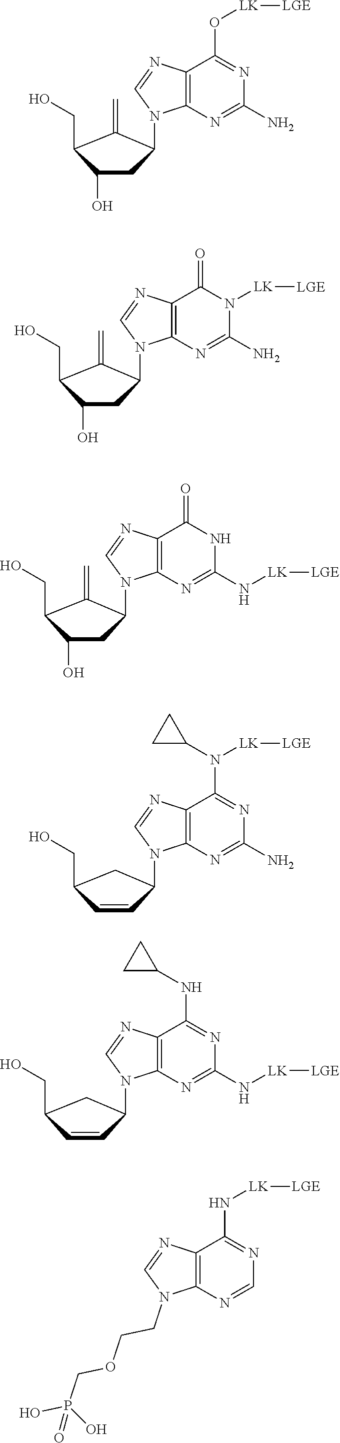

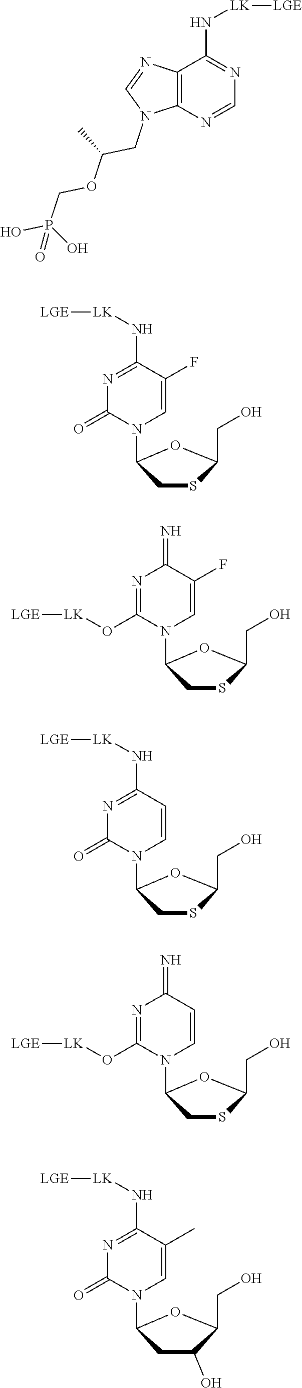

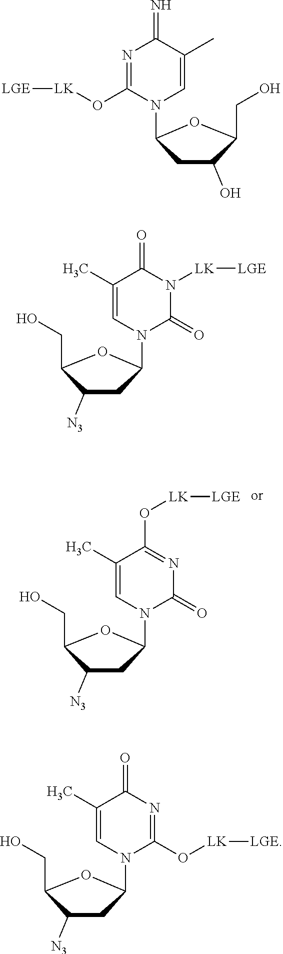

A fifth aspect of the present disclosure relates to the compound of LGP-LK-LGE provided above, wherein the ligand LGE binds to the E3 ubiquitin ligase is an optionally substituted structure shown below:

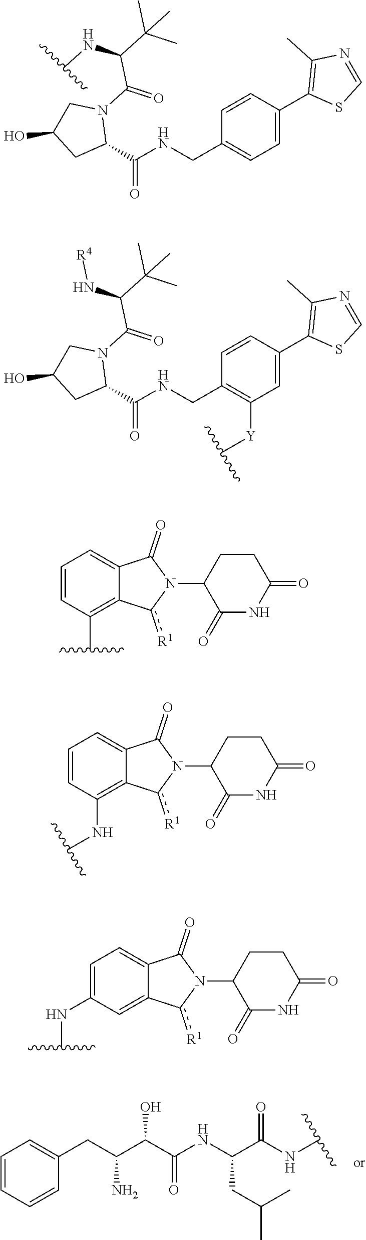

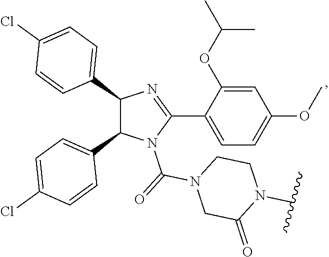

wherein, [symbol] represents the position linked to LK through a chemical bond; R¹ is optionally hydrogen, oxygen, C1-6 alkyl, C1-6 haloalkyl or alkoxy; R⁴ is optionally C1-6 alkyl, C1-6 haloalkyl or R⁵CO—; R⁵ is optionally C1-6 alkyl or C1-6 haloalkyl; Y is optionally C, O, S or NR⁶; and R⁶ is optionally C1-6 alkyl, C1-6 haloalkyl or alkoxy.

A sixth aspect of the present disclosure relates to the compound of LGP-LK-LGE provided above, wherein the linker LK linking the two parts LGP and LGE optionally has the following structure:

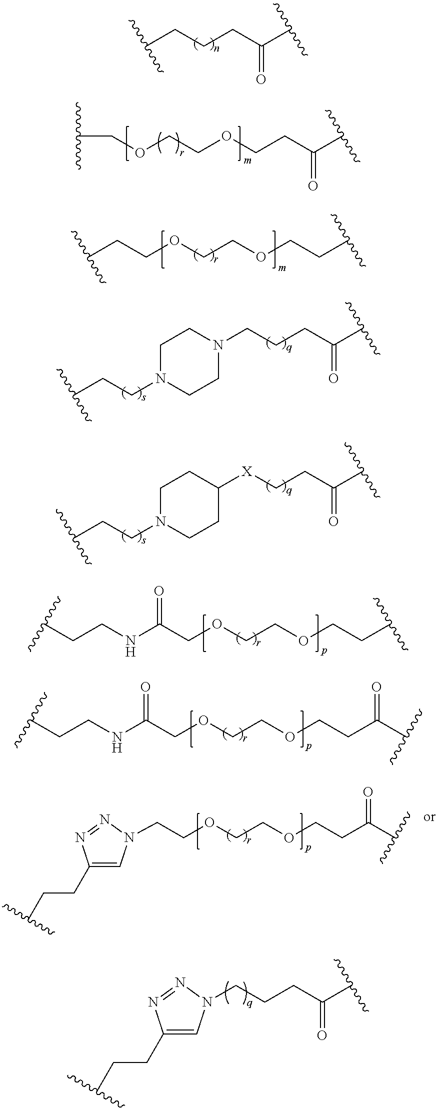

wherein, [symbol] represents the position where the linker LK linked with LGP or LGE through a chemical bond; m is optionally an integer of 0 to 5; n is optionally an integer of 0 to 25; p is optionally an integer of 0 to 4; q is optionally an integer of 0 to 20; r is optionally an integer of 1 to 3; s is optionally an integer of 1 to 5, X is optionally C, O, S or NR²; R² is optionally C1-6 alkyl, C1-6 haloalkyl or alkoxy.

A seventh aspect of the present disclosure relates to the compound of LGP-LK-LGE provided above, wherein the compound of LGP-LK-LGE optionally has the structure shown below:

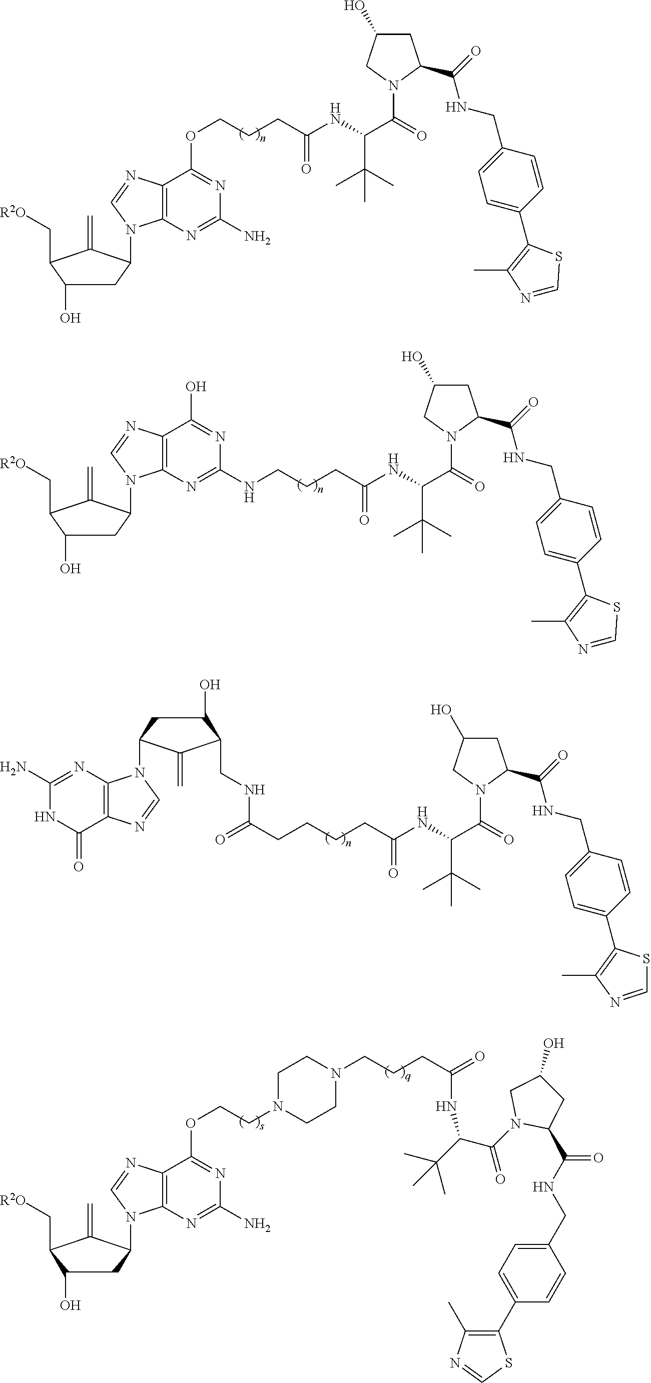

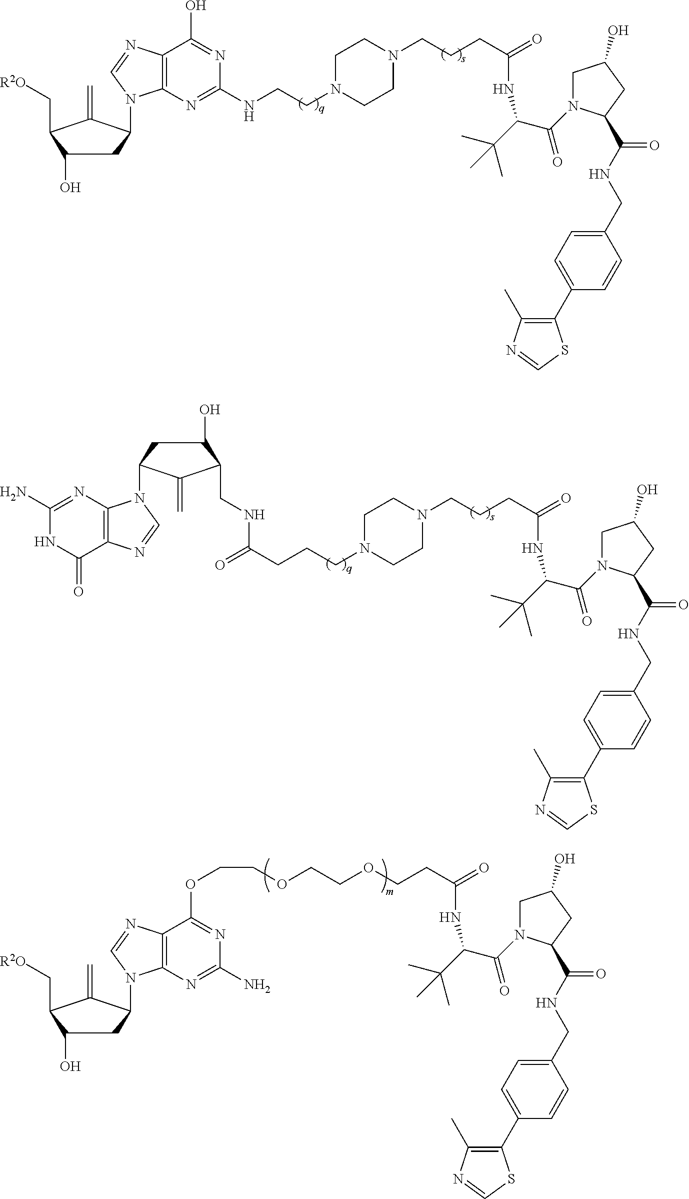

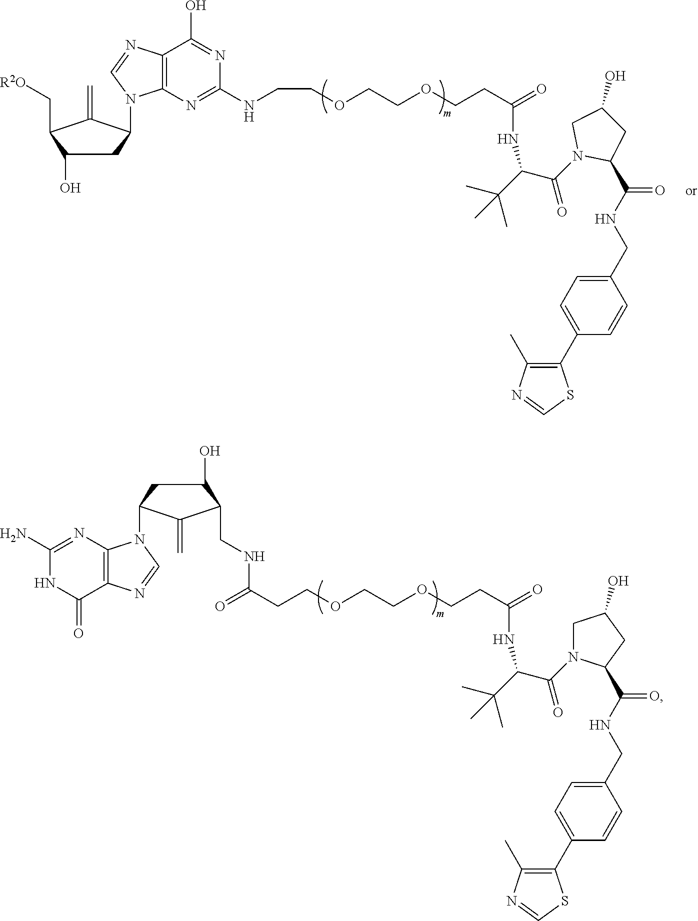

wherein, m is an integer of 0 to 5, n is an integer of 0 to 25, s is optionally an integer of 1 to 5, q is optionally an integer of 0 to 20, R² is optionally hydrogen, —P(O)(OH)₂, —P(O)OH—P(O)(OH)₂ or —P(O(OH)—P(O)(OH)—P(O)(OH)₂.

An eighth aspect of the present disclosure relates to the compound of LGP-LK-LGE provided above, wherein the compound of LGP-LK-LGE optionally has the structure shown below:

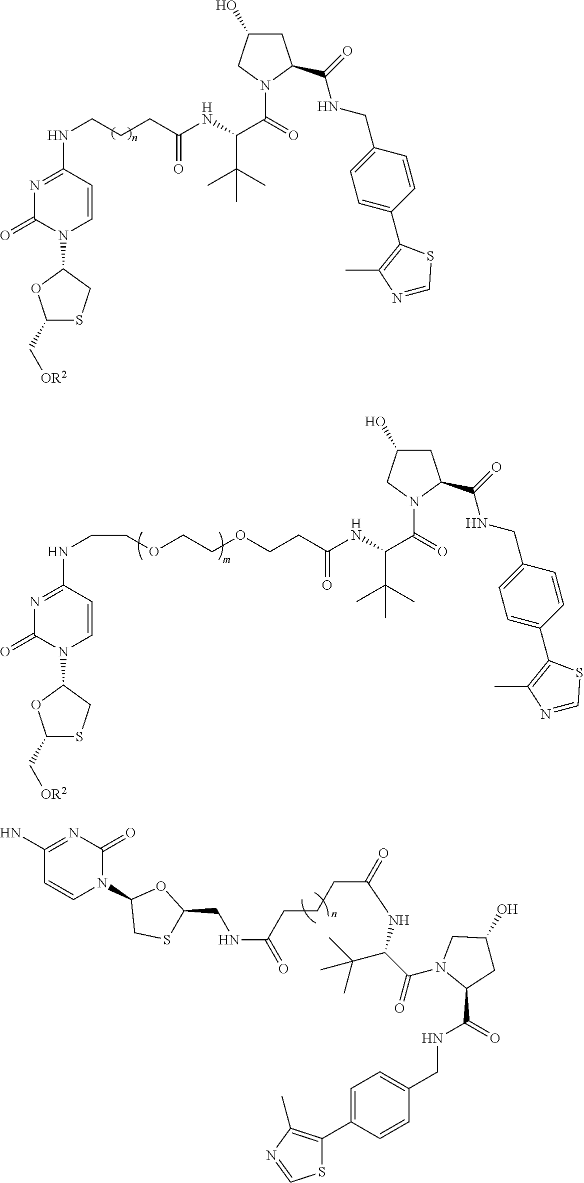

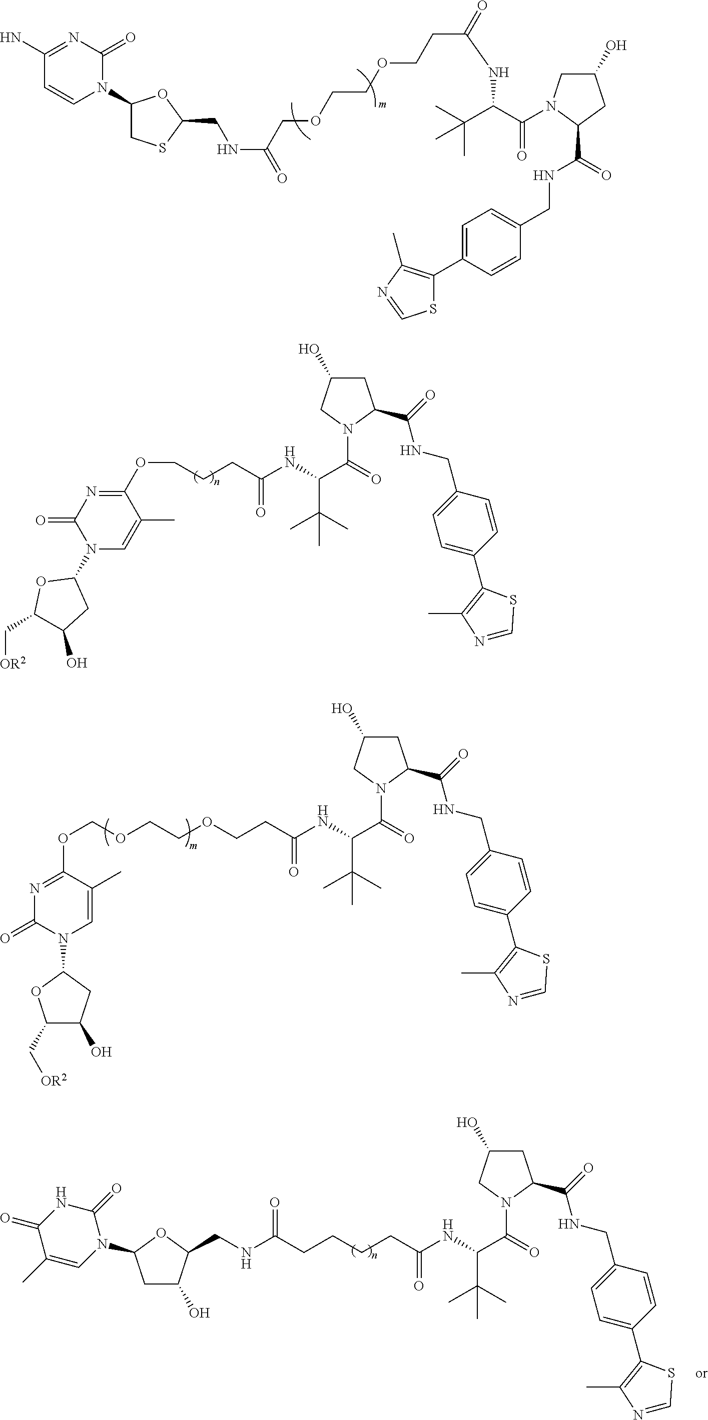

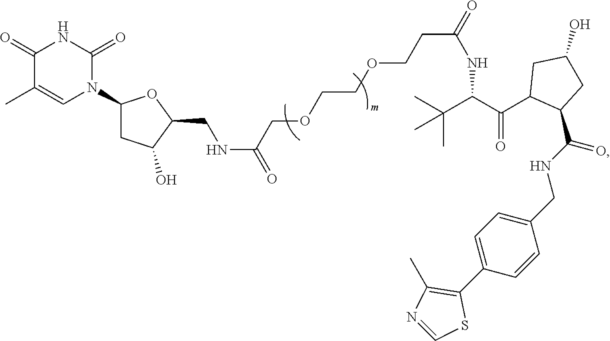

wherein, m is an integer of 0 to 5, n is an integer of 0 to 25, R² is optionally hydrogen, —P(O)(OH)₂, —P(O)OH—P(O)(OH)₂ or —P(O)(OH)—P(O)(OH)—P(O)(OH)₂; or the compound of LGP-LK-LGE optionally has the structure shown below:

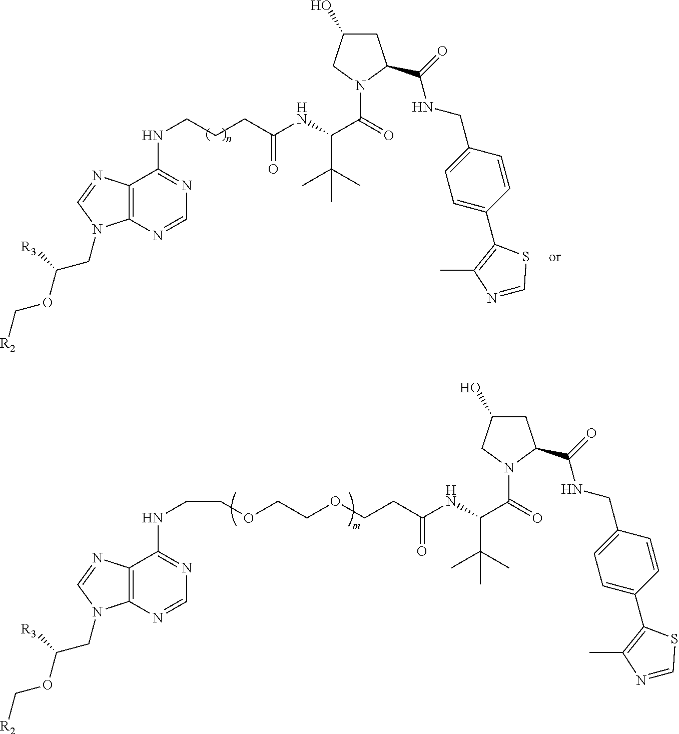

wherein, m is an integer of 0 to 5, n is an integer of 0 to 25, R² is optionally hydrogen, —P(O)(OH)₂, —P(O)OH—P(O)(OH)₂ or —P(O)(OH)—P(O)(OH)—P(O)(OH)₂; R³ is optionally hydrogen or methyl.

A ninth aspect of the present disclosure relates to the compound of LGP-LK-LGE provided above, wherein the compound of LGP-LK-LGE optionally has the structure shown below:

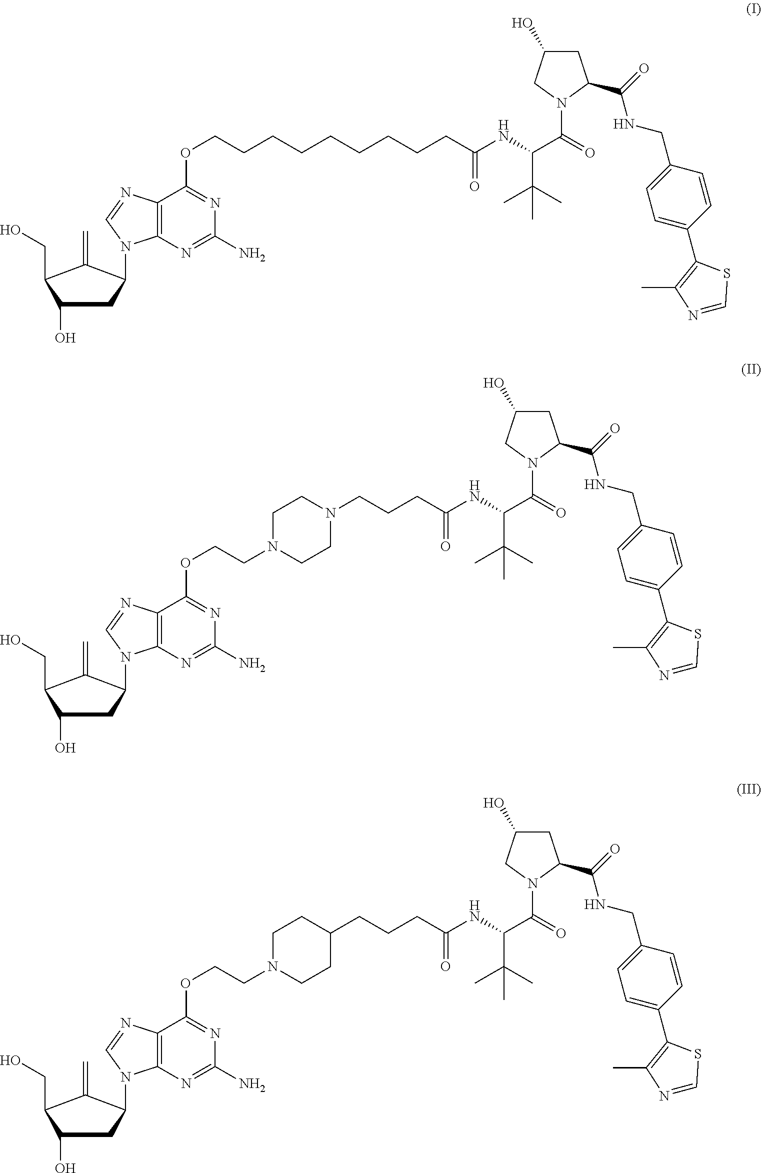 [SMILES: C=C1[C@H](CO)[C@H](O)C[C@@H]1n1cnc2c(OCCCCCCCCCC(=O)N[C@H](C(=O)N3C[C@H](O)C[C@H]3C(=O)NCc3ccc(-c4scnc4C)cc3)C(C)(C)C)nc(N)nc21 ; C=C1[C@H](CO)[C@H](O)C[C@@H]1n1cnc2c(OCCN3CCN(CCCC(=O)N[C@H](C(=O)N4C[C@H](O)C[C@H]4C(=O)NCc4ccc(-c5scnc5C)cc4)C(C)(C)C)CC3)nc(N)nc21 ; C=C1[C@H](CO)[C@H](O)C[C@@H]1n1cnc2c(OCCN3CCC(CCCC(=O)N[C@H](C(=O)N4C[C@H](O)C[C@H]4C(=O)NCc4ccc(-c5scnc5C)cc4)C(C)(C)C)CC3)nc(N)nc21]

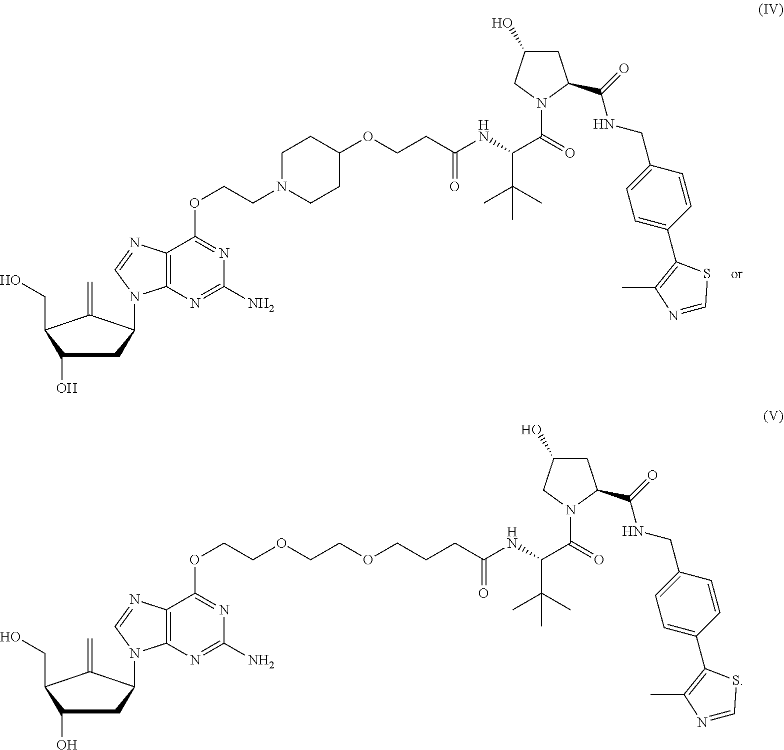 [SMILES: C=C1[C@H](CO)[C@H](O)C[C@@H]1n1cnc2c(OCCN3CCC(OCCC(=O)N[C@H](C(=O)N4C[C@H](O)C[C@H]4C(=O)NCc4ccc(-c5scnc5C)cc4)C(C)(C)C)CC3)nc(N)nc21 ; C=C1[C@H](CO)[C@H](O)C[C@@H]1n1cnc2c(OCCOCCOCCCC(=O)N[C@H](C(=O)N3C[C@H](O)C[C@H]3C(=O)NCc3ccc(-c4scnc4C)cc3)C(C)(C)C)nc(N)nc21]

A tenth aspect of the present disclosure relates to the compound of LGP-LK-LGE provided above, wherein the compound of LGP-LK-LGE optionally has the structure shown below:

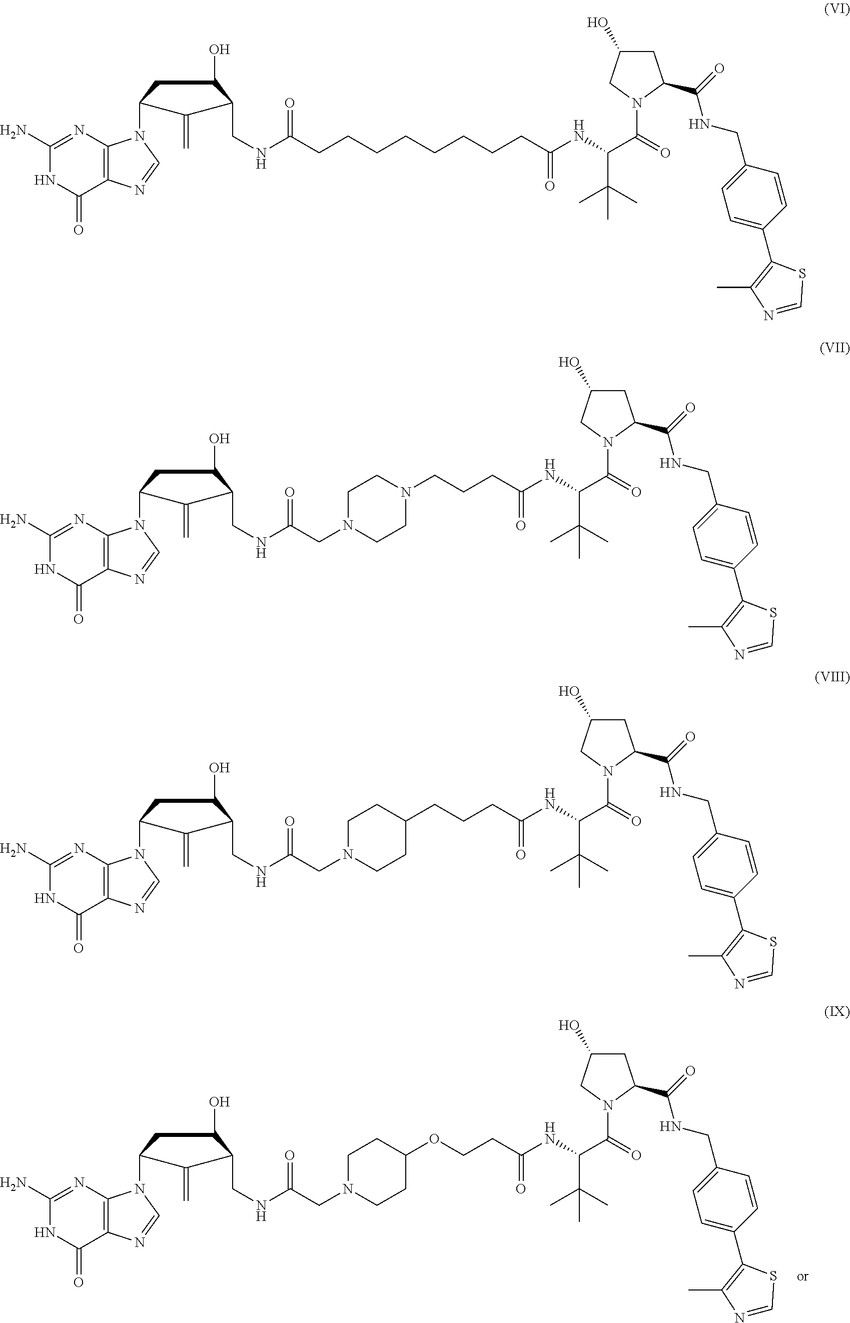

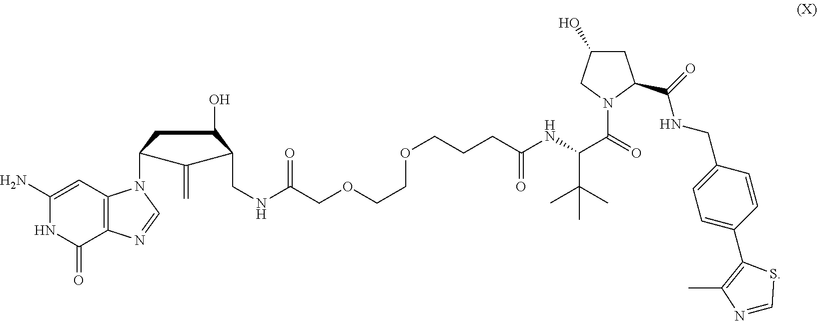 [SMILES: C=C1[C@H](CNC(=O)COCCOCCCC(=O)N[C@H](C(=O)N2C[C@H](O)C[C@H]2C(=O)NCc2ccc(-c3scnc3C)cc2)C(C)(C)C)[C@H](O)C[C@@H]1n1cnc2c(=O)[nH]c(N)cc21]

An eleventh aspect of the present disclosure provides a pharmaceutical composition, wherein the pharmaceutical composition comprises the compound of any one of the first aspect to the tenth aspect of the present disclosure.

A twelfth aspect of the present disclosure relates to the pharmaceutical composition of the eleventh aspect of the disclosure, wherein the pharmaceutical composition further comprises a pharmaceutically acceptable carrier, excipient, diluent, adjuvant, vehicle or a combination thereof.

A thirteenth aspect of the present disclosure relates to the pharmaceutical composition of the eleventh aspect of the disclosure, wherein, the administration route is at least one selected from the group consisting of nasal, inhalation, topical, oral, intramuscular, subcutaneous, transdermal, intraperitoneal, epidural, intrathecal and intravenous routes.

A fourteenth aspect of the disclosure relates to a preparation method of the compound described in any one of the first aspect to the tenth aspect of the present disclosure, wherein, the compound (I) can be optionally prepared from the following two synthetic routes:

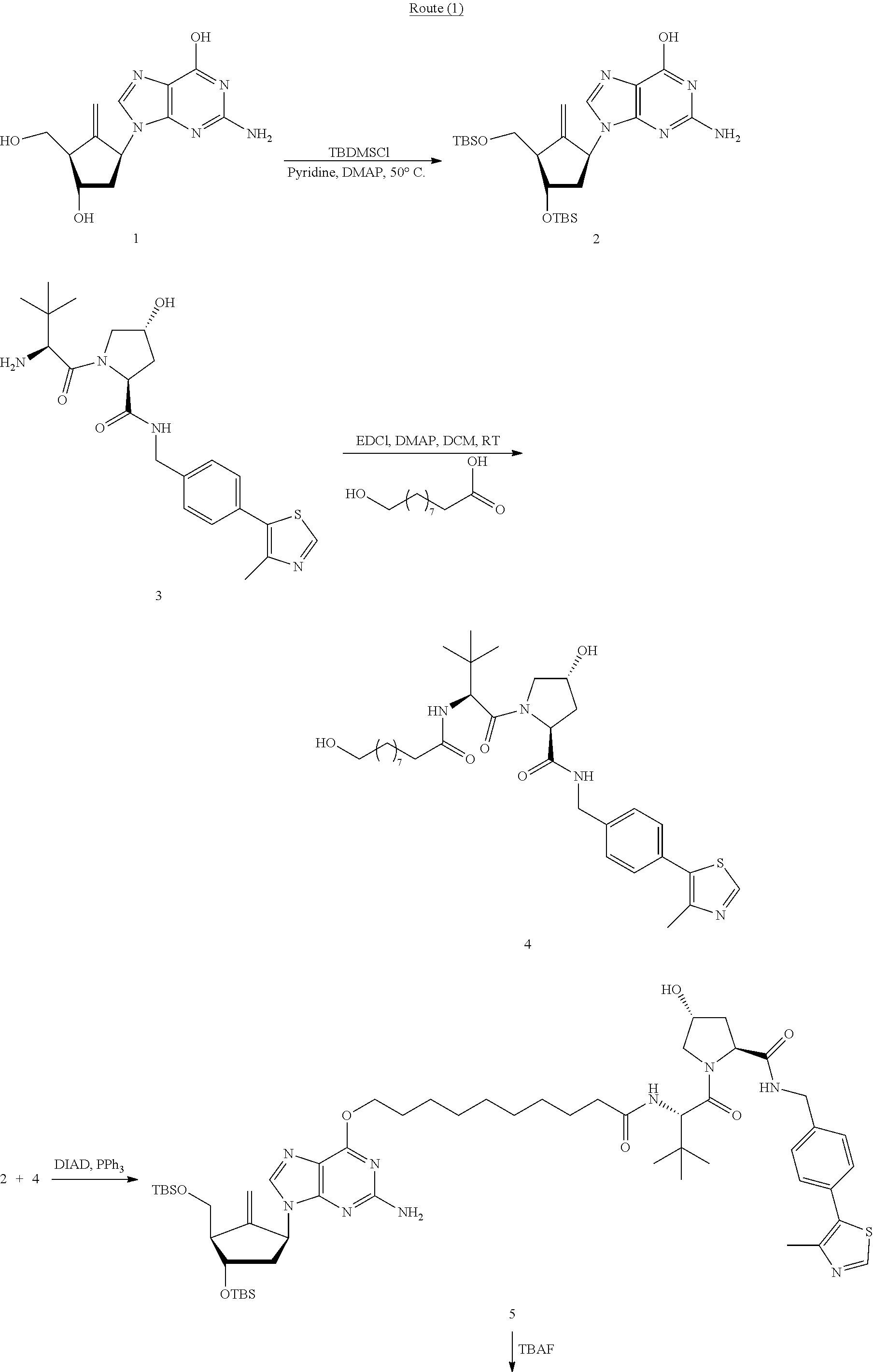

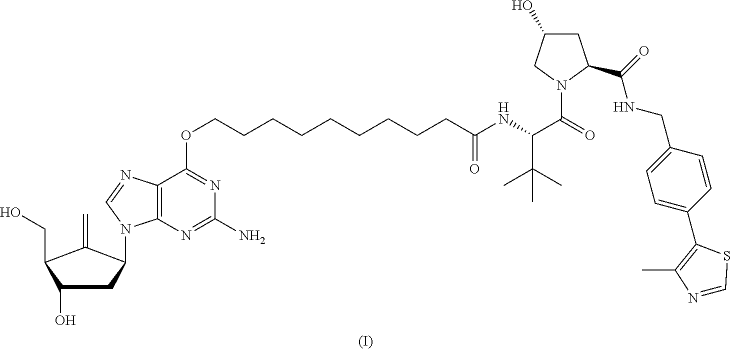

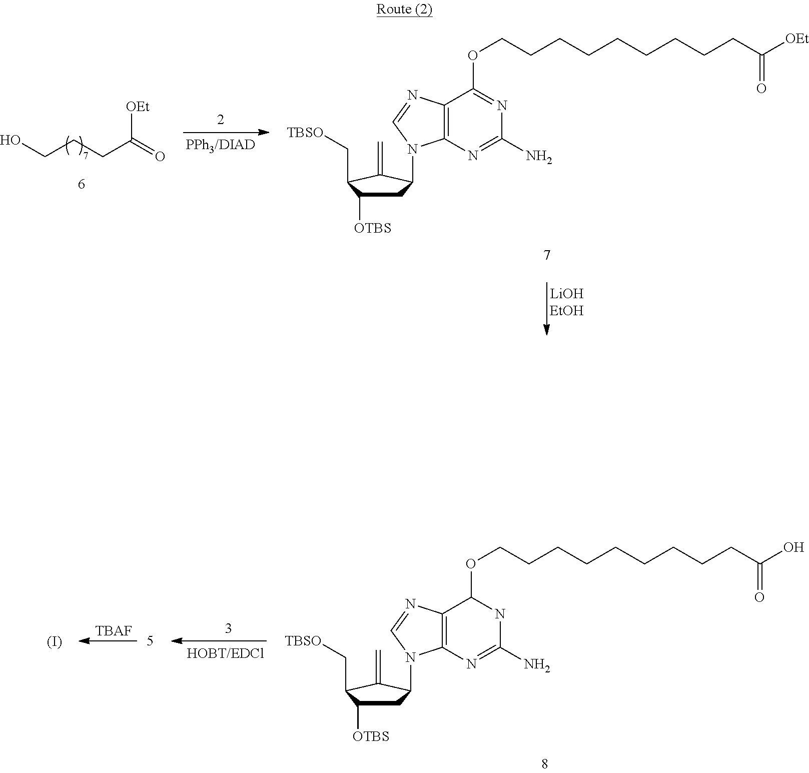

wherein, the compound (VI) can be prepared by the following synthetic route:

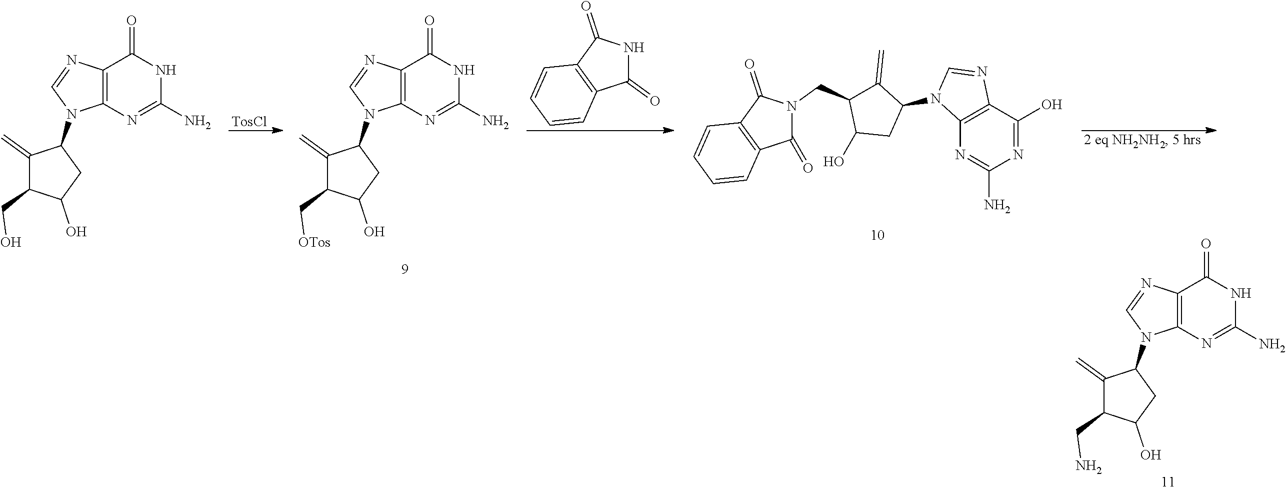

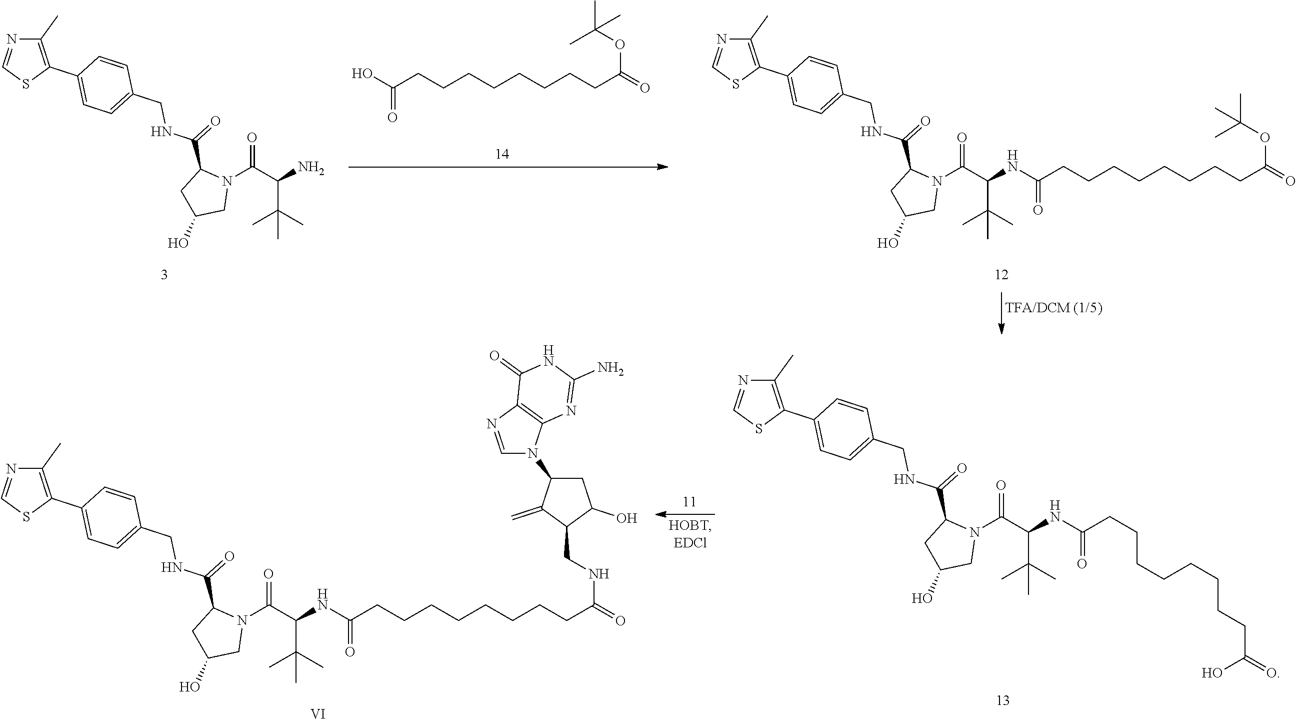

A fifteenth aspect of the present disclosure relates to the compound of any one of the first aspect to the tenth aspect of the present disclosure or the pharmaceutical composition of the eleventh aspect of the present disclosure, wherein the compound or the pharmaceutical composition can degrade and inhibit deoxyribonucleic acid (DNA) polymerase.

A sixteenth aspect of the present disclosure relates to the pharmaceutical composition of the eleventh aspect of the present disclosure, wherein the pharmaceutical composition can preferentially degrade and inhibit the virus endogenous deoxyribonucleic acid (DNA) polymerases.

A seventeenth aspect of the present disclosure relates to the pharmaceutical composition of the eleventh aspect of the present disclosure, wherein the pharmaceutical composition can more preferably degrade and inhibit hepadnaviridae virus endogenous deoxyribonucleic acid (DNA) polymerases.

An eighteenth aspect of the present disclosure relates to the pharmaceutical composition of the eleventh aspect of the present disclosure, wherein the pharmaceutical composition can more preferably degrade and inhibit hepatitis B virus endogenous deoxyribonucleic acid (DNA) polymerase.

A nineteenth aspect of the present disclosure relates to use of the compound of any one of the first aspect to the tenth aspect of the present disclosure or the pharmaceutical composition of the eleventh aspect of the disclosure for degrading and inhibiting deoxyribonucleic acid (DNA) polymerase.

A twentieth aspect of the present disclosure relates to use of the compound of any one of the first aspect to the tenth aspect of the present disclosure or the pharmaceutical composition of the eleventh aspect of the disclosure in the manufacture of a medicament for degrading and inhibiting deoxyribonucleic acid (DNA) polymerase.

A twenty-first aspect of the present disclosure relates to use of the compound of any one of the first aspect to the tenth aspect of the present disclosure or the pharmaceutical composition of the eleventh aspect of the disclosure for treating, preventing or diagnosing various diseases related to deoxyribonucleic acid polymerase, wherein the diseases include but are not limited to viral infectious diseases and secondary diseases thereof, the viral infectious diseases is infected by hepatitis B virus, human immunodeficiency virus, HCV, HDV, HEV, Ebola virus, SARS virus or COVID19; the secondary disease caused by the viral infection is liver cirrhosis, liver fibrosis, liver ascites or liver cancer.

A twenty-second aspect of the present disclosure relates to a cell line Huh7-HBP that simultaneously expresses LgBiT and HiBiT and highly expresses HBP gene, wherein, the cell line Huh7-HBP has HBP gene, and is preferably prepared by the following method:
(1) inserting LgBiT tag nucleotide sequence into the lentiviral vector pCDH-CMV-EFla-Neo to obtain a recombinant plasmid pCDH-CMV-LgBiT-EFla-Neo, carrying out lentivirus package on the recombinant plasmid pCDH-CMV-LgBiT-EFla-Neo together with lentivirus helper plasmids pMD2.G and pSPAX2, and transducing the packaged lentivirus into Huh7 cell line to obtain a new cell line Huh7-LgBiT;
(2) inserting the sequence of the target gene HBP with a HiBiT tag at the N-terminus into the lentiviral vector pLVX-Puro vector to obtain a recombinant plasmid pLVX-HBP-Puro, carrying out lentivirus package on the recombinant plasmid pLVX-HBP-Puro together with the lentiviral helper plasmids pMD2.G and pSPAX2, and transducing the packaged lentivirus into Huh7-LgBiT cell line to obtain the cell line Huh7-HBP which expresses both LgBiT and HiBiT and highly expresses the HBP gene.

A twenty-third aspect of the present disclosure relates to use of the cell line Huh7-HBP of the twenty-second aspect in determining the content of viral DNA polymerase in cells.

### BRIEF DESCRIPTION OF THE DRAWINGS

FIG. 1: ¹H NMR spectrum of the compound (I) of the present disclosure;

FIG. 2: Mass spectrum of the compound (I) of the present disclosure;

FIG. 3: High performance liquid chromatogram of the compound (I) of the present disclosure;

FIG. 4: ¹H NMR spectrum of compound (VI);

FIG. 5: Mass spectrum of compound (VI);

FIG. 6: High performance liquid chromatogram of compound (VI);

FIG. 7: The effect of compound (I) (TPD00203) on preventing virus replication in HepG2.2.15 cells (7 days);

FIG. 8: The effect of compound (I) (TPD00203) in preventing virus replication in HepAD38 cells (7 days);

FIG. 9: Comparison of compound (I) (TPD00203) in HepAD38 cells with the same concentration of positive control drug (ETV) in preventing virus replication (7 days);

FIG. 10: 36-48 hours experiment on the degradation of P protein by compound (I) (TPD00203) in Huh7 cells overexpressing Flag-polymerase;

FIG. 11: 30-36 hours experiment on the degradation of P protein by compound (I) (TPD00203) in Huh7 cells overexpressing Flag-polymerase;

FIG. 12: Plasmid sequence and map PLVX-HBP-HiBiT-Puro;

FIG. 13: Plasmid sequence and map pCDH-CMV-LgBiT-EFla-Neo;

FIG. 14: Protein degradation effect of compound (I) on HBP in Huh7-HBP cell line.

### DETAILED DESCRIPTION

The present disclosure is further described below through specific examples in conjunction with the accompanying drawings in the embodiments of the present disclosure. However, these examples are only used for more detailed description, and should not be construed to limit the present disclosure in any form. The present disclosure can be embodied in many different ways as defined and covered by the claims.

The present disclosure provides general and specific descriptions of the materials and experimental methods used in the experiments. Although many of the materials used and methods of operation for the purposes of the present disclosure are known in the art, the present disclosure is described herein in as much detail as possible. In the following, unless otherwise specified, the materials used and the methods of operation are well known in the art.

Unless otherwise defined, all terms (including technical and scientific terms) used in the present disclosure have the same meaning as commonly understood by one of those skilled in the art of the present disclosure.

### Example 1: Synthesis and Structural Confirmation of Compound (I) Targeting Degradation DNA Polymerase

The target compound (I, TPD00203) was prepared via the following synthetic route:

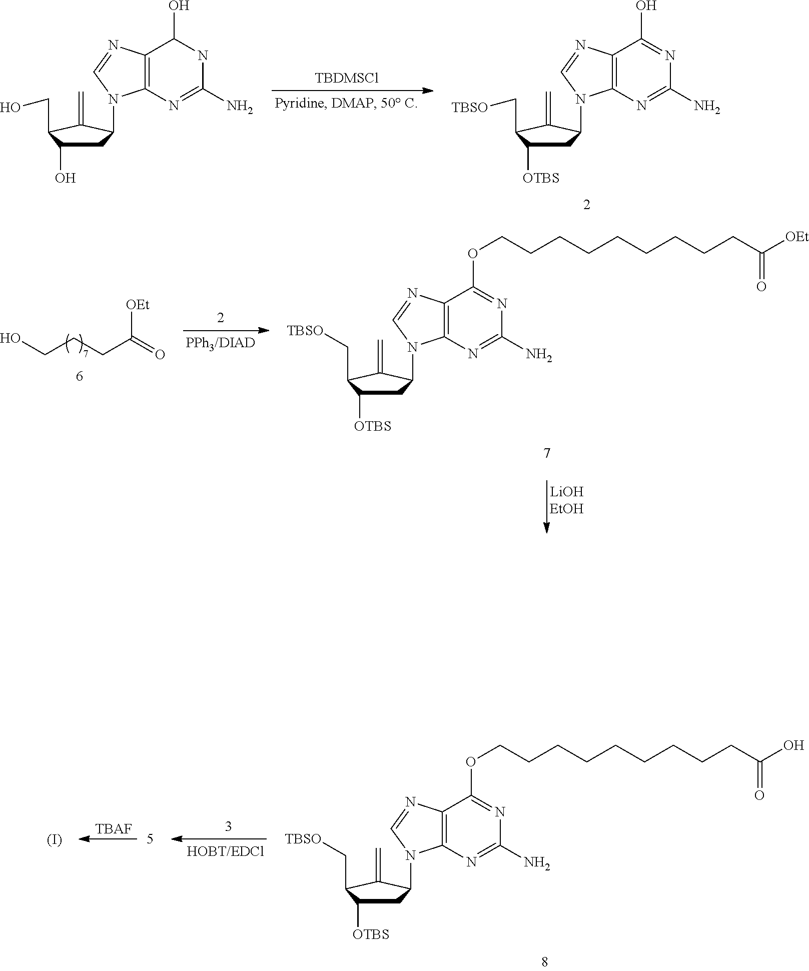

2-amino-9-[(1S,3S,4S)-4-tert-butyldimethylsiloxy-3-tert-butyldimethylsiloxymethyl-2-methylenecyclopentyl]-1,9-hydro-purin-6-ol (Compound 2): 2-amino-9-[(1S,3S,4S)-4-hydroxy-3-hydroxymethyl-2-methylenecyclopentyl]-1,9-hydro-purin-6-ol (700 mg, 2.52 mmol) and DMAP (16 mg, 0.13 mmol) were dissolved in pyridine (20 mL) at room temperature, and TBDMSCl (950 mg, 6.30 mmol) was slowly added at room temperature. The reaction solution was reacted at 50° C. for 24 hours. After cooling to room temperature, the reaction solution was poured into water, and extracted twice with ethyl acetate. The organic phase was washed twice with water, washed twice with saturated brine, dried with anhydrous sodium sulfate, and filtered. The filtrate was dried by rotary evaporation and separated on a preparative TLC plate to obtain 500 mg of compound 2 (yield: 39%). The product was a white solid.

Ethyl 10-((2-amino-9-[(1S,3S,4S)-4-tert-butyldimethylsiloxy-3-tert-butyldimethylsiloxymethyl-2-methylenecyclopentyl]-9-hydro-purin-6-oxodecanoate (Compound 7): Compound 2 (500 mg, 0.99 mmol) was dissolved in THF (10 mL) at room temperature, followed by adding ethyl 10-hydroxyldecanoate (Compound 6) (257 mg, 1.19 mmol), PPh₃ (391 mg, 1.49 mmol), DIAD (301 mg, 1.49 mmol). The reaction solution was replaced with nitrogen, reacted at room temperature for 24 hours, poured into water and extracted twice with ethyl acetate. The organic phase was washed twice with water, washed twice with saturated brine, dried over anhydrous sodium sulfate, and filtered. The filtrate was dried by rotary evaporation, and the residue was separated on a preparative TLC plate to obtain 430 mg of Compound 7 (yield: 62%).

10-(2-amino-9-[(1S,3S,4S)-4-tert-butyldimethylsiloxy-3-tert-butyldimethylsiloxymethyl)-2-methylenecyclopentyl]-9-hydro-purin-6-oxodecanoic acid (Compound 8): Compound 7 (430 mg, 0.61 mmol) was dissolved in EtOH (10 mL) at room temperature, and LiOH·H₂O (77 mg, 1.83 mmol) was added. The reaction solution was reacted at room temperature for 12 hours, and dried by rotary evaporation. The residue was dissolved in water, acidified to pH 4 with citric acid, and extracted twice with DCM. The organic phase was washed with water twice, dried over anhydrous sodium sulfate, and filtered. The filtrate was dried by rotary evaporation to obtain 370 mg of Compound 8 (yield: 90%).

(2S,4R)-1-((S)-2-(10-((2-amino-9-[(1S,3S,4S)-4-tert-butyldimethylsiloxy-3-tert-butyl dimethylsiloxymethyl-2-methylenecyclopentyl]-9-hydro-purin-6-alkyloxy)decanoylamide)-3,3-dimethylbutyryl)-N-(4-(4-methylthiazol-5-alkyl)benzyl)pyrrolidin-2-carboxamide (Compound 5): Compound 8 (370 mg, 0.54 mmol) and (2S,4R)-1-((S)-2-amino-3,3-dimethylbutyryl)-4-hydroxy-N-(4-(4-methylthiazol-5-alkyl)benzyl)pyrrolidin-2-carboxamide (compound 3, 252 mg, 0.54 mmol) was dissolved in DCM (10 mL) at room temperature, and successively added HOBT (109 mg, 0.81 mmol), EDCl (155 mg, 0.81 mmol), and DIEA (279 mg, 2.16 mmol). The reaction solution was reacted at room temperature for 12 hours. The reaction was quenched with water, and extracted twice with DCM. The organic phase was washed twice with water, dried over anhydrous sodium sulfate and filtered. The filtrate was dried by rotary evaporation and the resulted crude product was separated by preparative HPLC to obtain 230 mg of Compound 5 (yield: 39%).

(2S,4R)-1-((S)-2-(10-((2-amino-9-[(1S,3S,4S)-4-hydroxy-3-hydroxymethyl-2-methylenecyclopentyl]-9-hydro-purin-6-alkyloxy)decanoamide)-3,3-dimethylbutyryl)-N-(4-(4-methylthiazol-5-alkyl)benzyl) pyrrolidin-2-carboxamide (Compound I) (TPD00203): Compound 8 (230 mg, 0.21 mmol) was dissolved in THF (10 mL) at room temperature, TBAF (220 mg, 0.84 mmol) was added, and the reaction solution was reacted at room temperature for 12 hours. The organic phase was dried by rotary evaporation, and the crude product was separated by preparative HPLC to obtain 103 mg of white solid compound (I) (yield: 57%).

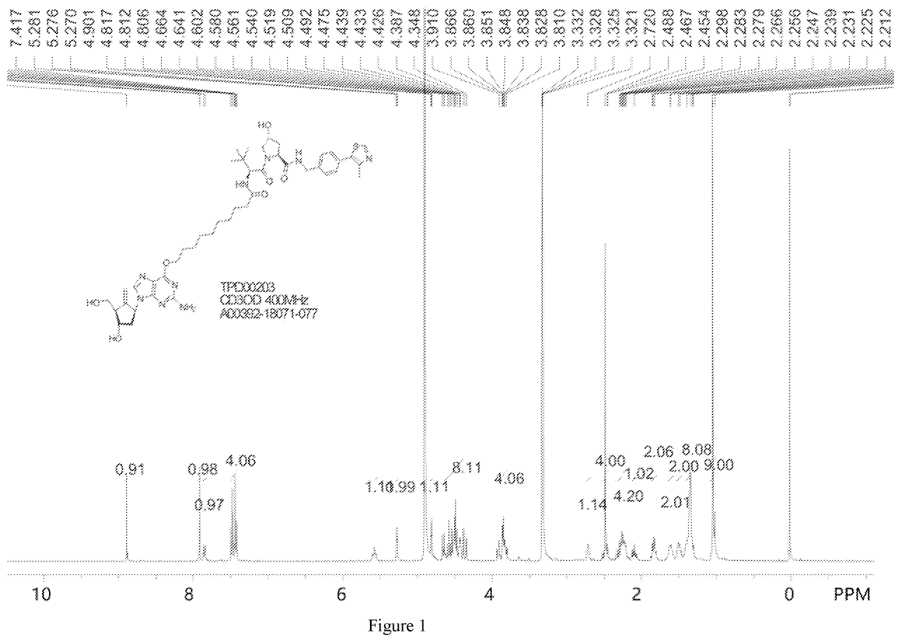

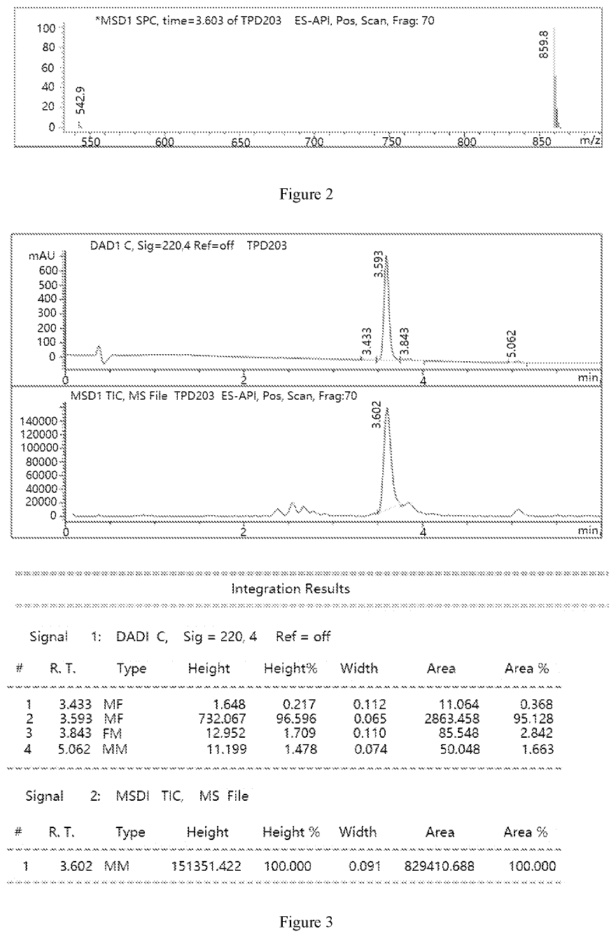

The structure and purity of the target compound (I, TPD00203) were confirmed by nuclear magnetic resonance spectroscopy, mass spectrometry and high performance liquid chromatography, and the purity of the expected compound (I, TPD00203) was confirmed to be higher than 95% by high performance liquid chromatography (see FIG. 1 to FIG. 3).

### Example 2: Synthesis and Structure Confirmation of Compounds II-V

2.1 The target compound II was prepared by the same synthetic method as compound I, except that the intermediate compound 6 used in the synthesis of compound I was replaced by the following intermediate:

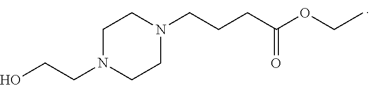 [SMILES: CCOC(=O)CCCN1CCN(CCO)CC1]

2.2 The target compound III was prepared by the same synthetic method as compound I, except that the intermediate compound 6 used in the synthesis of compound I was replaced by the following intermediate:

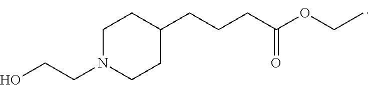 [SMILES: CCOC(=O)CCCC1CCN(CCO)CC1]

2.3 The target compound IV was prepared by the same synthetic method as compound I, except that the intermediate compound 6 used in the synthesis of compound I was replaced by the following intermediate:

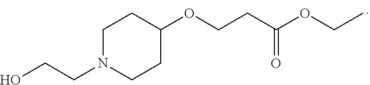 [SMILES: CCOC(=O)CCOC1CCN(CCO)CC1]

2.4 The target compound V was prepared by the same synthetic method as compound I, except that the intermediate compound 6 used in the synthesis of compound I was replaced by the following intermediate

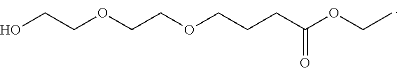

The structures of the above compounds were confirmed by nuclear magnetic resonance spectroscopy, mass spectrometry, and high resolution mass spectrometry; the purity was determined by high performance liquid chromatography. The data results were shown in the table of Example 5.

### Example 3: Synthesis and Structural Confirmation of Compound (VI) Targeting DNA Polymerase Degradation

The target compound (VI) was prepared via the following synthetic route

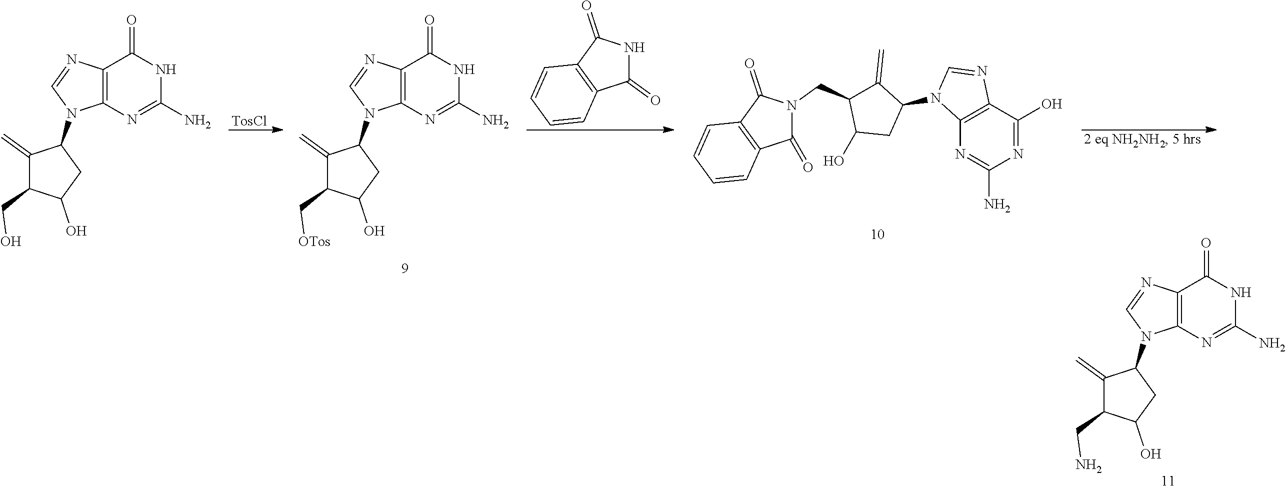

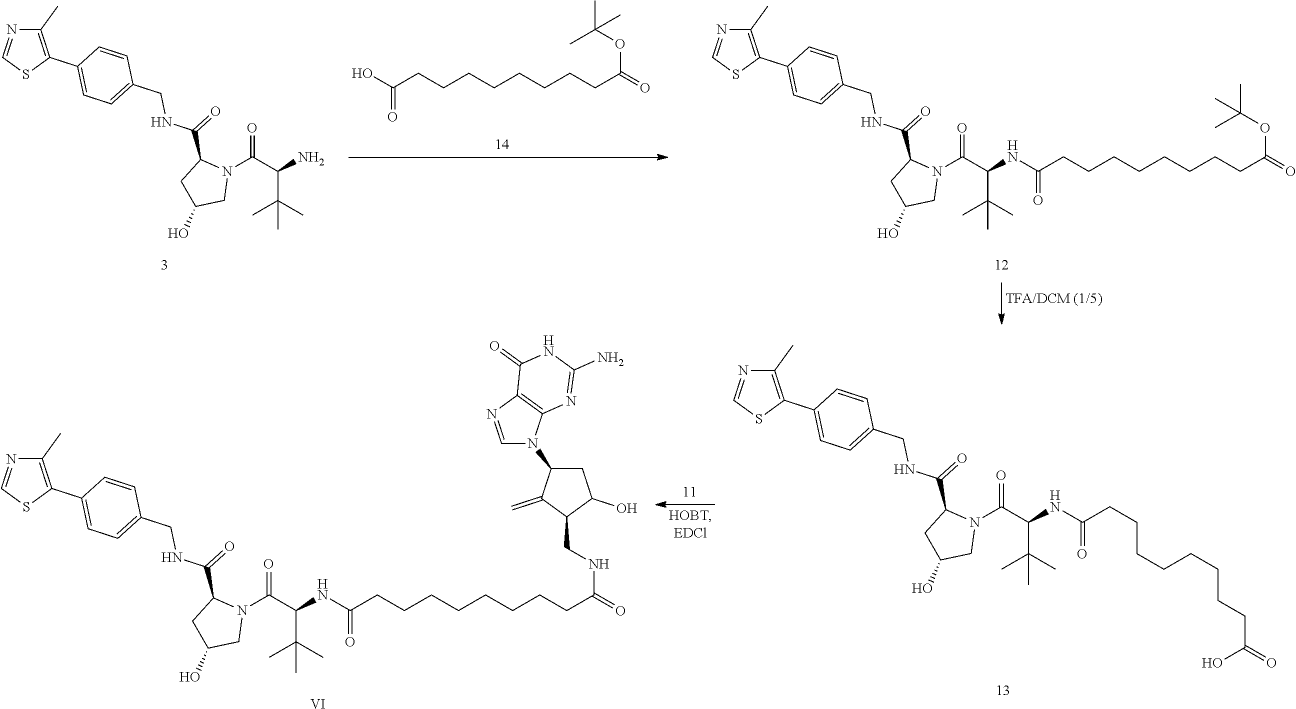

((1R,3S)-3-(2-Amino-6-oxo-1,6-dihydro-9H-purin-9-yl)-5-hydroxy-2-methylenecyclopentyl) methyl 4-methylbenzene sulfonate (compound 9): 2-amino-9-[(1S,3S,4S)-4-hydroxy-3-hydroxymethyl-2-methylenecyclopentyl]-1, 9-hydro-purin-6-ol (4 g, 14.4 mmol) and TosCl (3.57 g, 18.72 mmol) were stirred in pyridine (30 mL) at 25° C. overnight. After the reaction was completed, the reaction solution was concentrated under reduced pressure to obtain a yellow oily crude compound 9 (11.8 g, yield 57%). The crude product was directly used in the next reaction.

2-(((1R,3S)-3-(2-Amino-6-hydroxy-9H-purin-9-yl)-5-hydroxy-2-methylenecyclopentyl) methylisoindoline-1,3-dione (Compound 10): Compound 9 (11.8 g, 27.3 mmol), phthalimide (6.03 g, 40.9 mmol) and potassium carbonate (7.55 g, 54.6 mmol) were dissolved in DMF (110 mL), reacted at 50° C. for 16 hours. After the reaction was completed, filtration was performed to obtain a filtrate, and the filtrate was purified with prep-HPLC to obtain a gray solid compound 10 (0.32 g, purity 95%).

2-amino-9-((1S,3R)-3-(aminomethyl)-4-hydroxy-2-methylenecyclopentyl)-1,9-dihydro-6H-purin-6-one (Compound 11): Compound 10 (280 mg, 0.69 mmol) was dissolved in MeOH (5 mL), hydrazine hydrate (172.5 mg, 1.38 mmol) was added, and the reaction was carried out at 25° C. for 2 hours. After the reaction was completed, the reaction solution was poured into MTBE (30 mL), the product was precipitated, and the brown solid Compound 11 (150 mg, purity 90/a, yield 71%) was obtained by filtration.

Tert-butyl 10-(((S)-1-((2S,4R)-4-hydroxy-2-((4-(4-methylthiazol-5-yl)benzyl)carbamoyl) pyrrolidin-1-yl)-3,3-dimethyl-1-oxobutan-2-yl)amino)-10-oxodecanoate (Compound 12): (2S,4R)-1-((S)-2-amino-3,3-dimethylbutyryl)-4-hydroxy-N-(4-(4-methylthiazol-5-alkyl)benzyl) pyrrolidin-2-carboxamide (Compound 3, 200 mg, 0.46 mmol), 10-(tert-butoxy)-10-oxodecanoic acid (Compound 14, 144 mg, 0.56 mmol), HOBT (94.1 mg, 0.70 mmol), EDCl (133.6 mg, 0.70 mmol) and triethylamine (141 mg, 1.39 mmol) were dissolved in DMF (5 mL) and reacted at 25° C. for 16 hours. After the reaction was completed, the reaction solution was added to ice water (30 mL), and extracted with ethyl acetate (20 mL×2). The organic layer was dried over Na₂SO₄ and concentrated. The residue was separated on TLC plate to obtain yellow oily compound 12 (230 mg, purity 950, yield 70%).

10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecanoic acid (Compound 13): Compound 12 (200 mg, 0.30 mmol) was dissolved in DCM (5 mL), TFA (339.9 mg, 2.98 mmol) was added, and the reaction was carried out at 25° C. for 1 hour. After the reaction was completed, the solvent was dried by rotation evaporation to obtain a yellow oily compound 13 (150 mg, 95% purity, 68% yield).

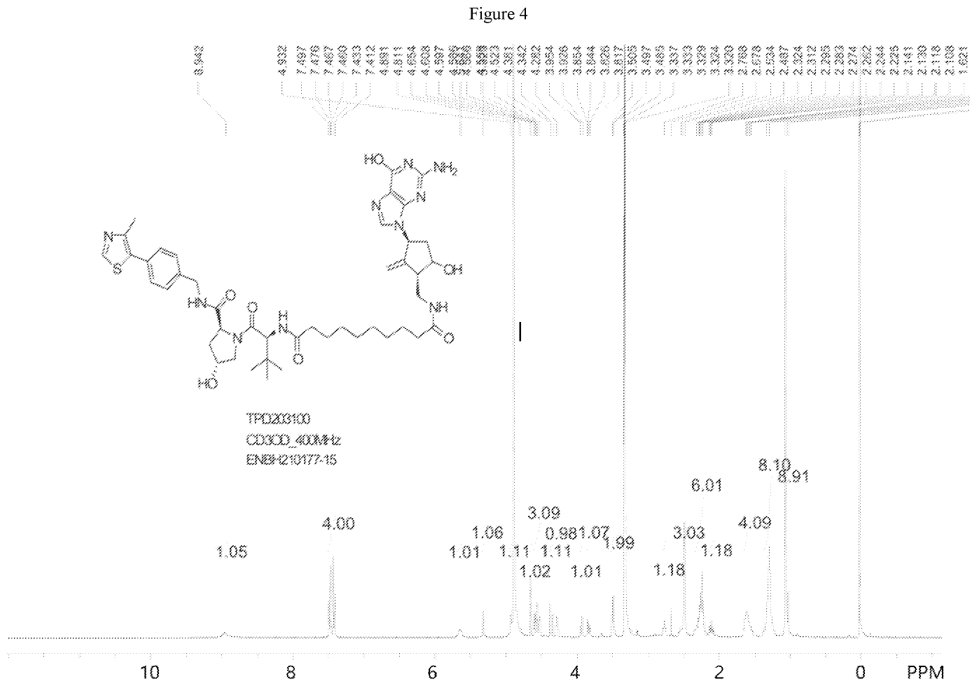

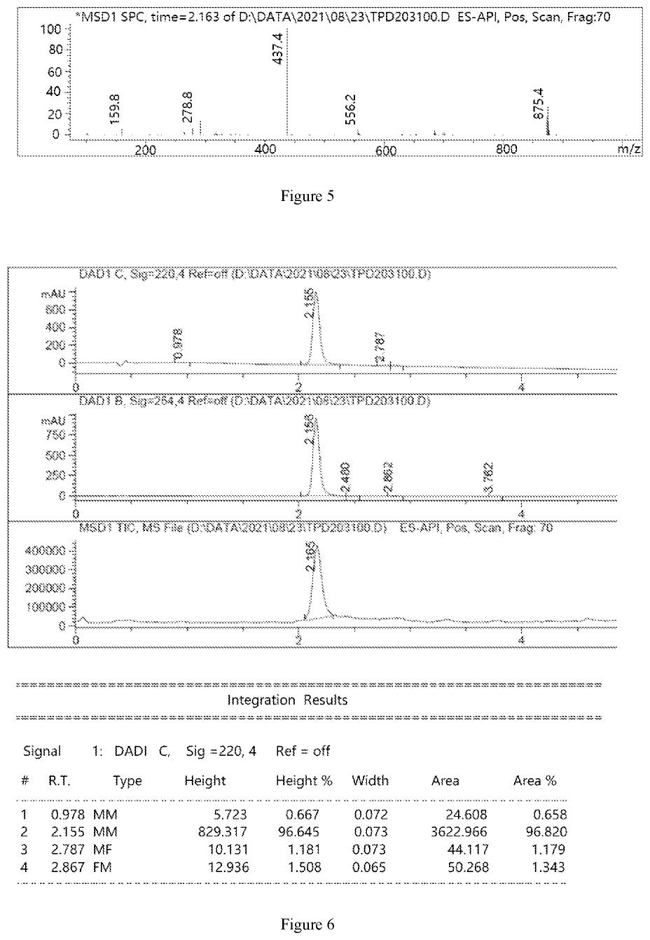

N1-(((1R,3S)-3-(2-amino-6-oxo-1,6-dihydro-9H-purin-9-yl)-5-hydroxy-2-methylenecyclopentyl) methyl)-N10-((S)-1-((2S,4R)-4-hydroxy-2-((4-(4-methylthiazol-5-yl)benzyl)carbamoyl) pyrrolidin-1-yl)-3,3-dimethyl-1-oxobutan-2-yl) decanoic acid diamide (Compound VI): Compound 13 (120 mg, 0.43 mmol) and Compound 11 (320.4 mg, 0.52 mmol) was dissolved in DMF (3 mL), HOBT (88.02 mg, 0.65 mmol), EDCl (124.9 mg, 0.65 mmol) and TEA (131.8 mg, 1.30 mmol) were added at room temperature, and the reaction solution was reacted at 25° C. for 16 hours. The reaction solution was purified with pre-HPLC to obtain a white solid (VI, 30 mg, purity 96%, yield 14%). The structure and purity of the target compound (VI) were confirmed by nuclear magnetic resonance spectroscopy, mass spectrometry and high performance liquid chromatography, and the purity of the expected compound (VI) was confirmed to be higher than 95% by high performance liquid chromatography (see FIG. 4 to FIG. 6).

### Example 4: Synthesis and Structure Confirmation of Compound VII-X

4.1 The target compound VII was prepared by the same synthetic method as compound VI, except that the intermediate compound 14 used in the synthesis of compound VI was replaced by the following intermediate:

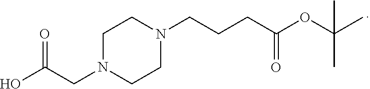 [SMILES: CC(C)(C)OC(=O)CCCN1CCN(CC(=O)O)CC1]

4.2 The target compound VIII was prepared by the same synthetic method as compound VI, except that the intermediate compound 14 used in the synthesis of compound VI was replaced by the following intermediate:

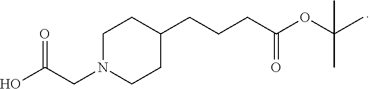 [SMILES: CC(C)(C)OC(=O)CCCC1CCN(CC(=O)O)CC1]

4.3 The target compound IX was prepared by the same synthetic method as compound VI, except that the intermediate compound 14 used in the synthesis of compound VI was replaced by the following intermediate:

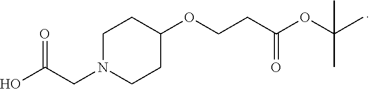 [SMILES: CC(C)(C)OC(=O)CCOC1CCN(CC(=O)O)CC1]

4.4 The target compound X was prepared by the same synthetic method as compound VI, except that the intermediate compound 6 used in the synthesis of compound VI was replaced by the following intermediate

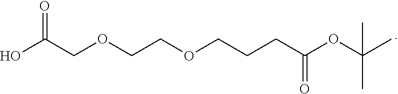 [SMILES: CC(C)(C)OC(=O)CCCOCCOCC(=O)O]

The structures of the above compounds were confirmed by nuclear magnetic resonance spectroscopy, mass spectrometry, and high resolution mass spectrometry; the purity was determined by high performance liquid chromatography. The data results were shown in the table of Example 5.

### Example 5: The Results of Mass Spectrometry, High Resolution Mass Spectrometry and High Performance Liquid Chromatography Analysis of Compound I-X

| | | | Calculated | Measured | |
|---|---|---|---|---|---|
| | Liquid | | value of high | value of high | |
| | chromatography - | | resolution mass | resolution mass | Liquid |
| | mass | Molecular | spectrum | spectrum | chromatography |
| Compound | spectrometry | formula | (M + 1)⁺ | (M + 1)⁺ | (Purity) |
| I | 859.8 | C₄₄H₆₁N₉O₇S | 860.4487 | 860.4572 | >95% |
| II | 889.1 | C₄₄H₆₁N₁₁O₇S | 888.4549 | 888.4956 | >95% |
| III | 888.1 | C₄₅H₆₂N₁₀O₇S | 887.4596 | 887.4677 | >95% |
| IV | 890.3 | C₄₄H₆₀N₁₀O₈S | 889.4389 | 889.4197 | >95% |
| V | 866.1 | C₄₂H₅₇N₉O₉S | 864.4073 | 865.0112 | >95% |
| VI | 875.4 | C₄₄H₆₀N₁₀O₇S | 873.4440 | 873.4395 | >95% |
| VII | 902.5 | C₄₄H₆₀N₁₂O₇S | 901.4501 | 901.4792 | >95% |
| VIII | 901.9 | C₄₅H₆₁N₁₁O₇S | 900.4549 | 901.0007 | >95% |
| IX | 903.1 | C₄₄H₅₉N₁₁O₈S | 902.4342 | 902.6765 | >95% |
| X | 878.2 | C₄₂H₅₆N₁₀O₉S | 877.4025 | 877.3866 | >95% |

### Example 6 Inhibition Experiment of Compound (I, TPD00203) on HBV Virus Replication in Different Cell Lines

In vitro cell model 1: HepG2 cells transfected with hepatitis B virus (HBV), namely HepG2 2.2.15 cells.

In vitro cell model 2: Stable toxigenic HepAD38 cells.

Positive drug control: entecavir (ETV).

Experimental procedure: HepG2.2.15 cells and stable toxigenic HepAD38 cells were divided into six experimental groups. The number of cells in each well was 7×10⁴, and the amount of medium in each well was 500 μl. The first group was a blank control, the second group was a positive control, and 3.75 nM ETV was added; the third to sixth groups were added with 3.75 nM, 100 nM, 5 μM and 100 μM compound (I, TPD00203). The supernatant was collected on the 3rd day after administration and the HBV DNA level in the supernatant was detected. On the seventh day, the supernatant was collected again and the cells were pelleted, and the level of HBV DNA in the supernatant was detected.

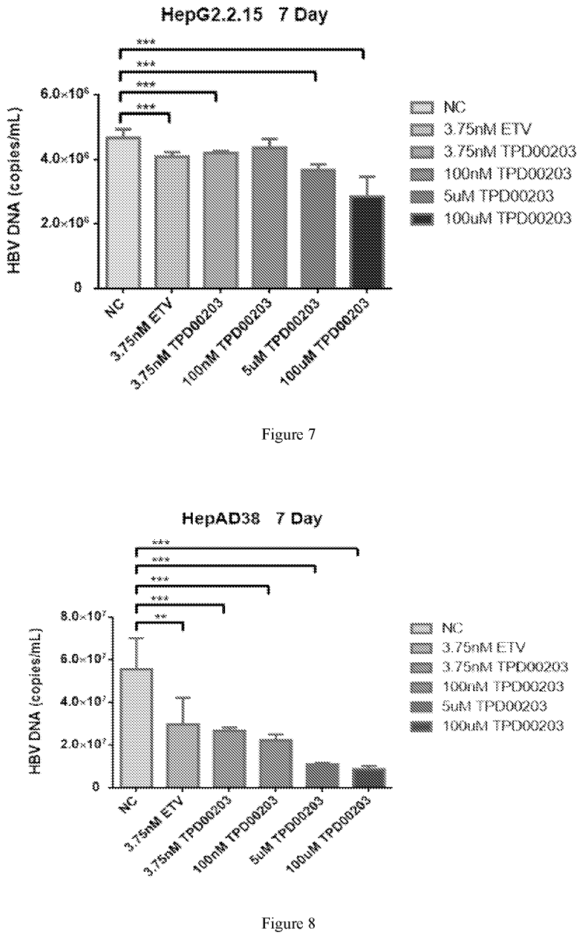

Experimental results: the inhibitory effects on HBV replication were shown in FIGS. 7-8. In the HepG2.2.15 cell line, after 7 days of administration, the entecavir group showed a significant inhibitory effect on virus replication (P≤0.001). Compared with the blank group, the TPD00203 administration group of 3.75 nM (P≤0.001), 5 μM (P≤0.001) and 100 μM (P≤0.001) all three doses showed significant inhibition of viral replication (FIG. 7). In the HepAD38 cell line, each dose group and the positive control group showed a significant inhibitory effect on virus replication after 7 days of administration (FIG. 8).

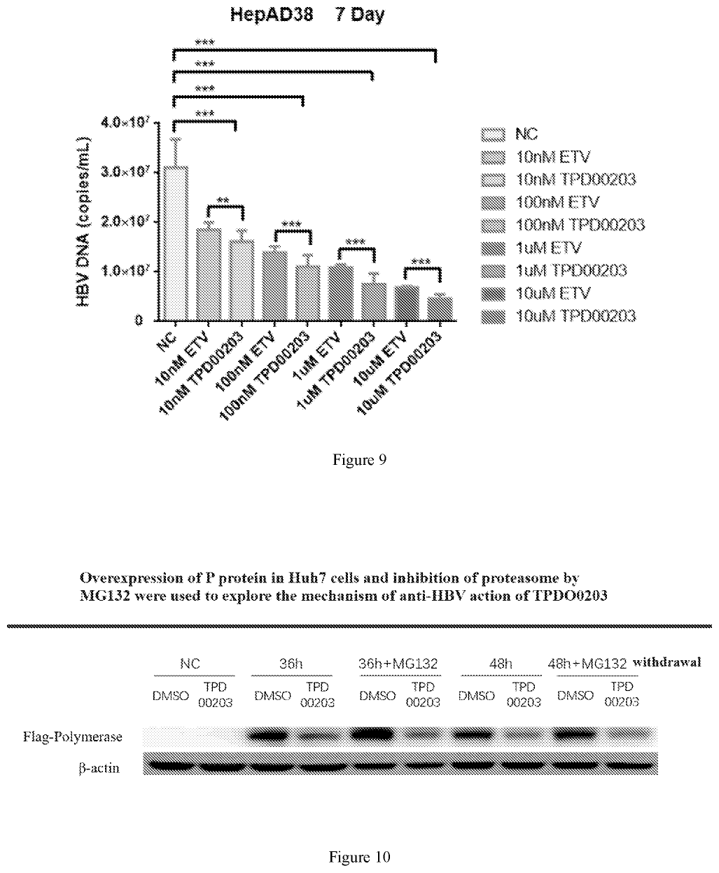

After the preliminary evaluation of the virus-inhibiting effect of TPD00203, the inhibitory effects of the same dose of TPD00203 and ETV were determined in HepAD38 cell line. ETV and TPD00203 were 4 dose groups of 10 nM, 100 nM, 1 μM, and 10 μM. The 7-day experimental results showed that both ETV and TPD00203 could significantly inhibit virus replication at all tested dose levels (P≤0.001), and TPD00203 had better virus-inhibiting effect than ETV at the same dose (FIG. 9).

### Example 7 the Results of Inhibitory Activity of Compound (I-X) of HBV Virus Replication in HepAD38 Cell Line

The inhibitory activity of compounds (I-X) on the virus in HepAD38 cell line was determined by the same method as in Example 6, and the results were shown in the following table.

Antiviral activity of the compound
at different concentrations *

| Compound | 10 nM | 100 nM | 1000 nM | 10000 nM |
|---|---|---|---|---|
| I | +++ | +++ | +++ | +++ |
| II | + | + | ++ | +++ |
| III | +++ | +++ | +++ | +++ |
| IV | +++ | +++ | +++ | +++ |
| V | + | ++ | ++ | ++ |
| VI | + | ++ | +++ | +++ |
| VII | + | + | ++ | ++ |
| VIII | ++ | ++ | ++ | +++ |
| IX | ++ | ++ | +++ | +++ |
| X | + | + | + | ++ |

* +++ exhibits >60% inhibitory activity against viral replication; ++ exhibits 30-60% inhibitory activity against viral replication; + exhibits 10-30% inhibitory activity against viral replication

### Example 8 the Degradation Effect of Compound (I, TPD00203) on HBV P Protein in Huh7 Cell Line

In vitro cell model: Huh7.

Proteasome inhibitor: MG132

The Huh7 cells cultured in the medium containing 10 μM TPD00203 drug were used to transfect the flag-tagged P protein overexpression plasmid, and the medium containing 10 μM TPD00203 drug was changed 6 hours after transfection, and MG132 (final concentration 10 μM) was added 24 hours after transfection to inhibit the degradation of P protein, and cells were harvested after MG132 treatment for 12 hours. MG132 is a reversible proteasome inhibitor. After MG132 was removed, the cells were cultured in a medium containing 10 μM TPD00203 for 12 hours, and the cells were harvested. The expression of P protein was detected by Western Blot experiment. DMSO was used as a control.

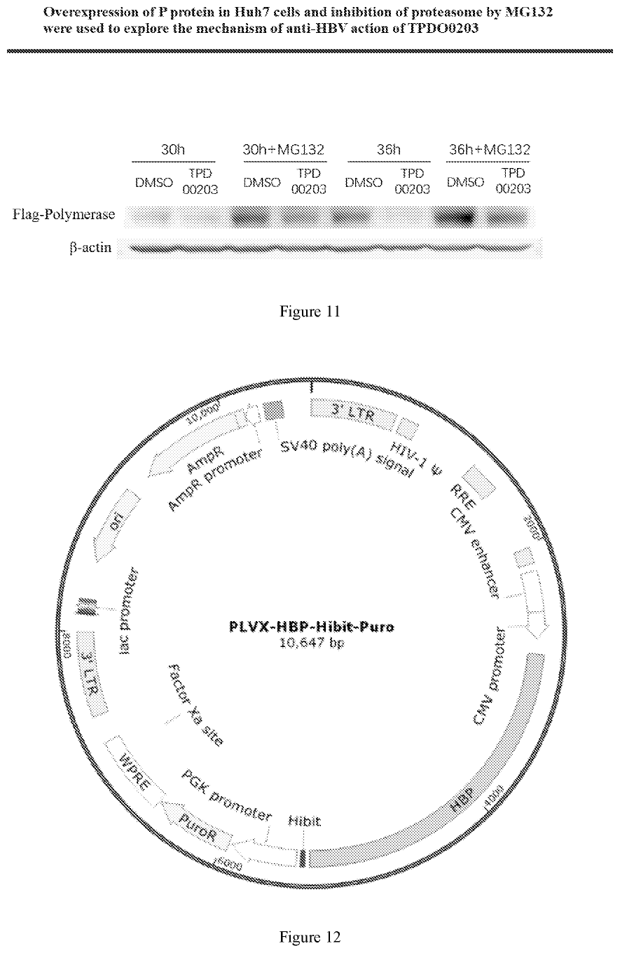

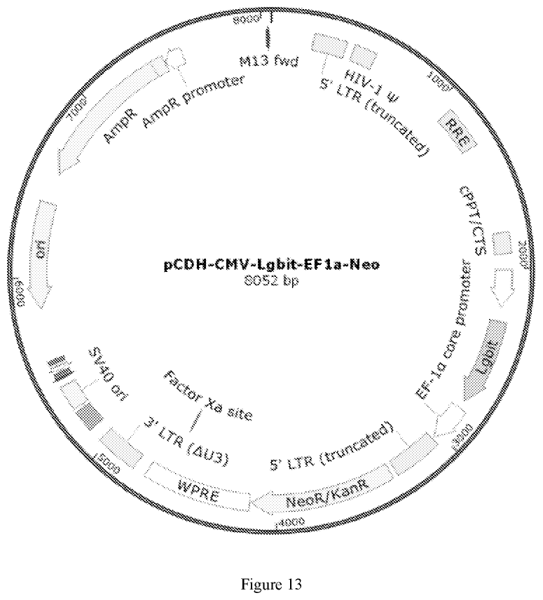

Experimental results: Huh7 cells were transfected with flag-Polymerase plasmid, and the proteasome inhibitor MG132 was added 24 hours after transfection to inhibit the degradation of P protein. TPD00203 was observed to promote P protein degradation 36 hours after transfection, and MG132 inhibited drug degradation. The P protein was degraded rapidly, and the level of P protein expression at 48 hours after transfection was lower than that at 36 hours after transfection. The proteasome inhibition effect of MG132 is reversible, and the P protein accumulated in the cells will continue to be degraded after drug withdrawal (FIG. 10-FIG. 11).

### Example 9 Construction of a Cell Line (Huh7-HBP) Expressing LgBiT and HiBiT while Highly Expressing HBP Gene

### Cell Line Construction Process:

9.1 Obtaining the target plasmid pCDH-CMV-LgBiT-EFla-Neo

First, the LgBiT tag nucleotide sequence was obtained by gene synthesis, and both ends of the sequence had Nhe I and BamH I restriction enzyme cutting sites. After the sequence was synthesized, the sequence was inserted into the lentiviral vector pCDH-CMV-EFla-Neo by double digestion and ligation, and the recombinant plasmid was named pCDH-CMV-LgBiT-EFla-Neo. The recombinant plasmid adopts the CMV promoter and carries the neomycin resistance gene.

9.2 lentiviral packaging

The target plasmid pCDH-CMV-LgBiT-EFla-Neo together with the lentiviral helper plasmids pMD2.G and pSPAX2 were used for lentiviral packaging. The lentivirus packaging process is as follows:

The 293FT cell culture flask (T175) that has grown to 80%-90% was taken out from the cell culture incubator at 37° C. with 5% CO₂, 2 mL of TrypLE™ EXPRESS was added to digest and then the cells was collected, washed, and re-plated in a 145 mm plate, 20 mL DMEM medium (Thermo Fisher) was added, and shaken gently so that the cells cover approximately 80% of the plate, and cultured in a 37° C., 5% CO₂ incubator.

After 24 hours, the three plasmids were mixed with the transfection reagent PEI-Pro (polyplus, Art. No.: 29031C1B), and allowed to stand at room temperature for 10 min. The 293FT cells used for virus packaging were taken out from the cell culture incubator at 37° C. with 5% CO₂, the above mixture was added to each plate evenly, shaken gently, and placed in a 37° C., 5% CO₂ incubator. After 4 hours, the old medium was discarded, 5 mL of pre-warmed PBS was added to wash the cells, then 20 mL of fresh pre-warmed DMEM medium containing 10% fetal bovine serum was added, and cultured in a 37° C. 5% CO₂ incubator.

After culturing for 48 h-72 h, the culture supernatant was collected as the virus stock solution. The stock solution was centrifuged at high speed for 2 h. The supernatant was discarded and the virus particles were resuspended in serum-free medium. Volume ratio of medium added to virus stock solution is 1:500. This is a virus concentrate. The virus concentrate was divided into 100 μl/tube, and another 10 μl was reserved for virus titer determination. The aliquoted concentrate was stored at −80° C.

9.3 Construction of Huh7-LgBiT cell line

Antibiotic resistance tests were conducted first before constructing positive cell lines. DMEM+10% FBS complete medium containing different concentrations of G418 (MCE, HY-17561) was added to the 24-well plate plated with Huh7 cell line. When the concentration of G418 reached 300 μg/ml, all Huh7 cells died. This concentration was proved to be the maximum tolerated concentration of blank Huh7, and the subsequent positive cell lines were screened with this concentration.

Transduction of the packaged lentivirus into the Huh7 cell line:
Day 1: The Huh7 cell line was plated in 6-well plate according to 2E5/well;
Day 2: LgBiT lentivirus was added according to 20 μl/well and mixed gently;
Day 3: The transduced cell line was added according to the pre-tested antibiotic concentration, and conducted a blank Huh7 control at the same time.

When all the cells in the control group died and the cells in the experimental group were still alive, the screening was stopped. The cell lines of the experimental group continued to be cultured, and 300 μg/ml of G418 was added at the same time.

9.4 Construction of PLVX-HBP-Puro plasmid

First, the sequence of the target gene HBP with a HiBiT tag at the N-terminus was obtained by gene synthesis, and the two ends have Xho I and BamH I restriction enzyme cutting sites.

After the sequence synthesis was completed, the sequence was inserted into the lentiviral vector pLVX-Puro vector by double digestion and ligation. The recombinant plasmid was named PLVX-HPP-Puro. The recombinant plasmid adopts the CMV promoter and carries the puromycin resistance gene.

9.5 lentiviral packaging

The target plasmid pLVX-HBP-Puro together with the lentiviral helper plasmids pMD2.G and pSPAX2 were used for lentiviral packaging. The packaging process is the same as 9.2.

9.6 Huh7-HBP

Antibiotic resistance tests were conducted first before constructing positive cell lines. DMEM+10% FBS complete medium containing different concentrations of puromycin (InvivoGen, ant-pr-1) was added to the 24-well plate plated with Huh7-LgBiT cell line. When the concentration of puromycin reached 2 μg/ml, all Huh7-LgBiT cells died. This concentration was proved to be the maximum tolerated concentration of Huh7-LgBiT, and subsequent positive cell lines were screened with this concentration.

Transduction of the packaged lentivirus into the Huh7-LgBiT cell line:
Day 1: The Huh7-LgBiT cell line was plated in 6-well plates according to 2E5/well;
Day 2: HBP-HiBiT lentivirus was added according to 20 μl/well and mixed gently;
Day 3: The transduced cell line was added according to the pre-tested antibiotic concentration, and conducted Huh7-LgBiT control at the same time.

When all the cells in the control group died and the cells in the experimental group were still alive, the screening was stopped. The cell lines in the experimental group continued to be cultured, and 2 μg/ml of puromycin was added at the same time. Finally, a cell line Huh7-HBP that expresses both LgBiT and HiBiT and highly expresses HBP gene was obtained.

HBP sequence reference: UniProt ID P03156.

LgBiT sequence:
SEQ ID No. 1
atggtcttcacactcgaagatttcgttggggactgggaacagacag
ccgcctacaacctggaccaagtccttgaacagggaggtgtgtcca
gtttgctgcagaatctcgccgtgtccgtaactccgatccaaagga
ttgtccggagcggtgaaaatgccctgaagatcgacatccatgtca
tcatcccgtatgaaggtctgagcgccgaccaaatggcccagatcg
aagaggtgtttaaggtggtgtaccctgtggatgatcatcacttta
aggtgatcctgccctatggcacactggtaatcgacggggttacgc
cgaacatgctgaactatttcggacggccgtatgaaggcatcgccg
tgttcgacggcaaaaagatcactgtaacagggaccctgtggaacg
gcaacaaaattatcgacgagcgcctgatcacccccgacggctcca
tgctgttccgagtaaccatcaacagttga

HiBiT sequence is from Promega, and available by viewing and agreeing to the terms of use at https://promega.formstack.com/forms/hibit_synthesis_licensing_agreement

### Example 10 the Degradation Effect of Compound (I-X) on HBVP Protein in the Constructed Huh7-HBP Cell Line

### I: Test Steps for Determining HBVP Protein Concentration in Huh7-HBP Cell Line:

The transfected huh-7-HBP cell line was digested and suspended, 100 μl of cell suspension was added to each well of a 96-well plate at a concentration of 2×10⁵/ml, and incubated overnight. 10 μl of drug was added to each well according to the pre-set dose, and each dose of each drug should be at least two replicate wells. After shaking gently, the cell suspension was put back into the incubator, and incubated for 1-24 h according to the specific situation. Nano-Glo@ LCS Dilution Buffer and Nano-Glo@ Live Cell Substrate two reagents in the Nano-Glo@Live Cell Assay System kit were mixed at a ratio of 20:1, and added to the cell plate to be tested at a ratio of 25 ul per 100 ul system. After shaking for 30 seconds, full wavelength luminosity was measured using BMG Labtech FLUOstar Omega ELIASA. The higher the luminescence value, the higher the HBP content of the target protein.

II: Degradation Test Results of HBVP Protein (i.e. DNA Polymerase) in Huh7-HBP Cell Line:

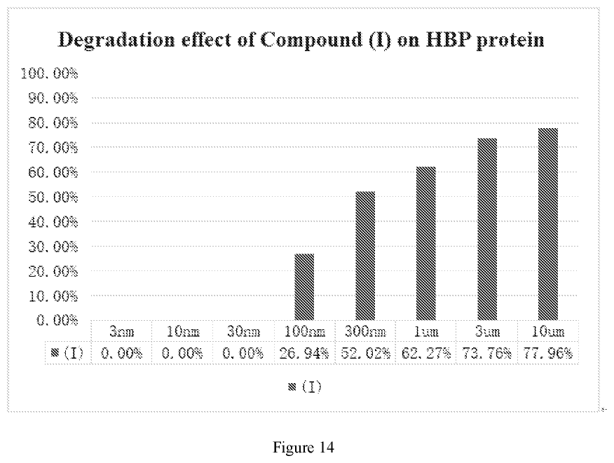

According to method 1, the degradation effect of target compounds I-X on HBV P protein at 5 different concentrations was determined. Compounds I-X all showed different degrees of P protein degradation effect. See FIG. 14 and the table below.

| | Degradation activity of DNA polymerase at | | | | |
|---|---|---|---|---|---|
| | different concentrations of compounds * | | | | |
| Compound | 100 nM | 300 nM | 1000 nM | 3000 nM | 10000 nM |
| I | + | ++ | ++ | +++ | +++ |
| II | + | + | + | ++ | ++ |
| III | + | + | ++ | ++ | +++ |
| IV | + | + | + | ++ | ++ |
| V | + | ++ | ++ | ++ | ++ |
| VI | + | ++ | ++ | +++ | +++ |
| VII | + | + | + | ++ | ++ |
| VIII | + | + | ++ | ++ | +++ |
| XI | + | ++ | ++ | ++ | +++ |
| X | + | + | + | ++ | ++ |

* +++ protein degradation >70%; ++ protein degradation 30-70%; + protein degradation <30%

In conclusion, the present disclosure provides a novel PROTAC compound that can effectively degrade HBV DNA polymerase. The experimental results show that these compounds can prevent viral replication by degrading the DNA polymerase of the virus, and provide a solution for the effective treatment of viral infections.

1. A bifunctional compound and a pharmaceutically acceptable salt, a solvate, a hydrate, a polymorph, a tautomer, a geometric isomer, an isotopic label, a metabolite or a prodrug thereof, wherein, the bifunctional compound has the formula of LGP-LK-LGE, wherein LGP is a ligand binding deoxyribonucleic acid (DNA) polymerase, LGE is a ligand of E3 ubiquitin ligase, and LK is a linker linking LGP and LGE.

2. The bifunctional compound of claim 1, wherein the LGP is selected from the group consisting of the following structures, or is selected from the structures phosphorylated, bisphosphorylated or triphosphorylated at a hydroxyl group of any of the following structures:

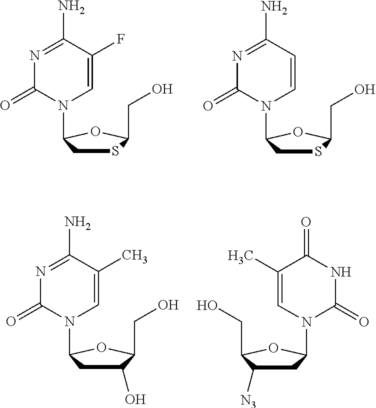

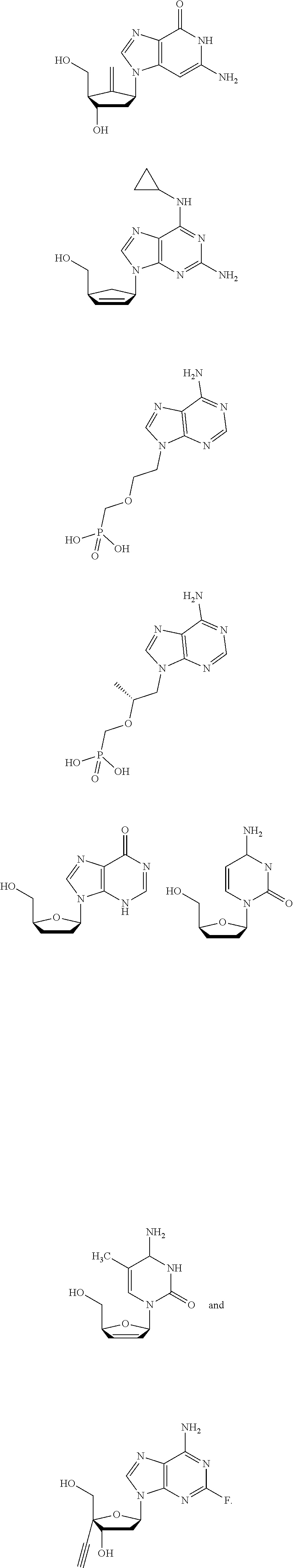

3. The bifunctional compound of claim 1, wherein the LGE is selected from the group consisting of: 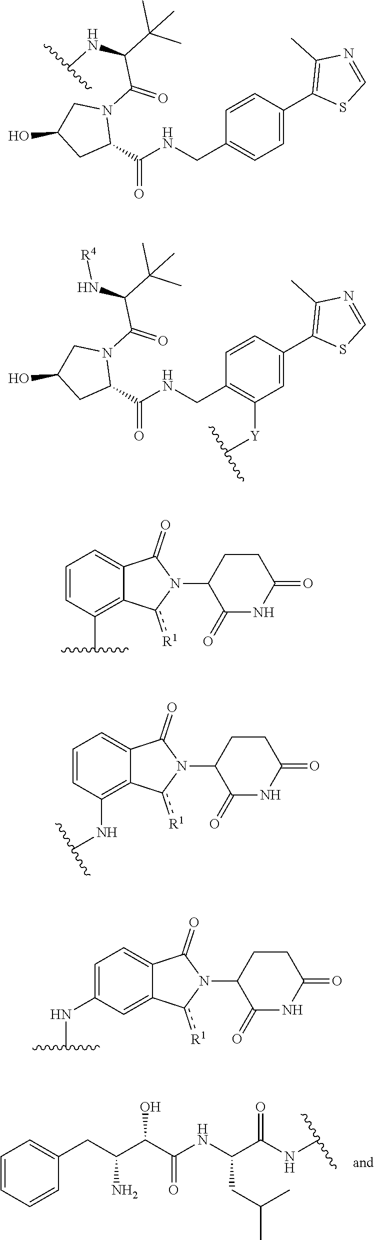 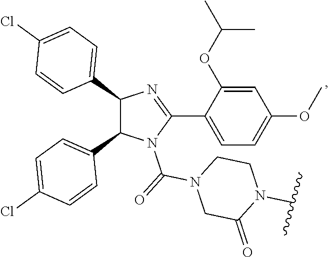
wherein, [symbol] represents the position linked to LK through a chemical bond; R¹ is optionally hydrogen, oxygen, C1-6 alkyl, C1-6 haloalkyl or alkoxy; R⁴ is optionally C1-6 alkyl, C1-6 haloalkyl or R⁵CO—; R⁵ is optionally C1-6 alkyl or C1-6 haloalkyl; Y is optionally C, O, S or NR⁶; and R⁶ is optionally C1-6 alkyl, C1-6 haloalkyl or alkoxy.

4. The bifunctional compound of claim 1, wherein the LK optionally has the following structure: 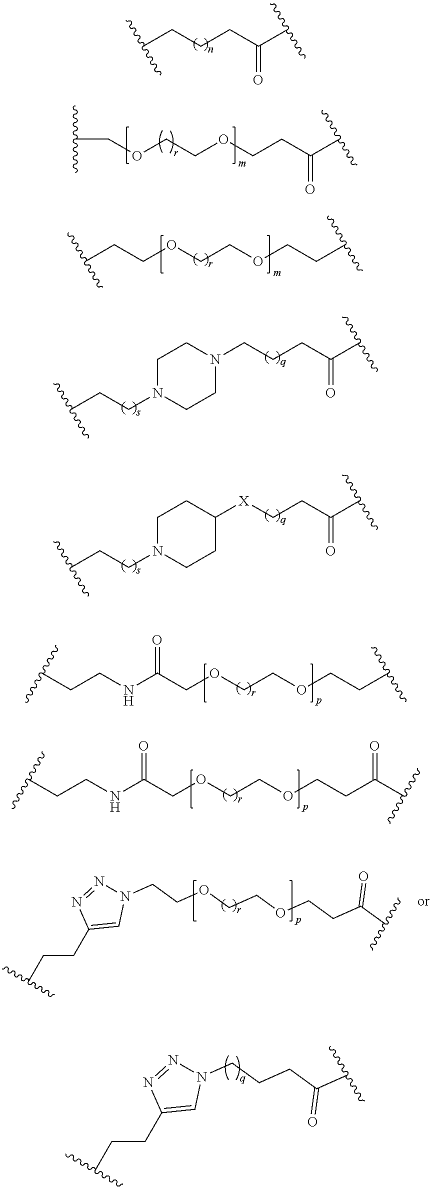
wherein, [symbol] represents the position where the linker LK linked with LGP or LGE through a chemical bond; m is optionally an integer of 0 to 5; n is optionally an integer of 0 to 25; p is optionally an integer of 0 to 4; q is optionally an integer of 0 to 20; r is optionally an integer of 1 to 3; s is optionally an integer of 1 to 5, X is optionally C, O, S or NR²; R² is optionally C1-6 alkyl, C1-6 haloalkyl or alkoxy; preferably, the LK is linked to LGP through a chemical bond; more preferably, the LK is linked to the base of LGP, further preferably, the linking position is as shown in an of the following structures: 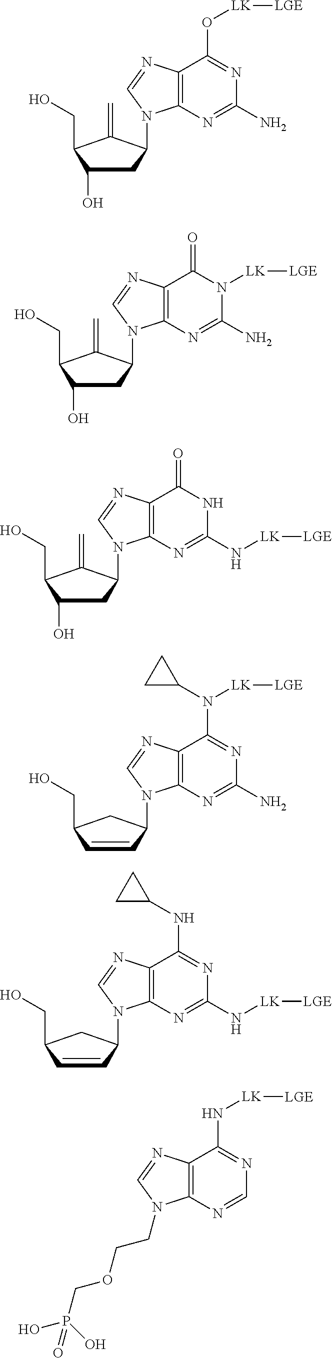 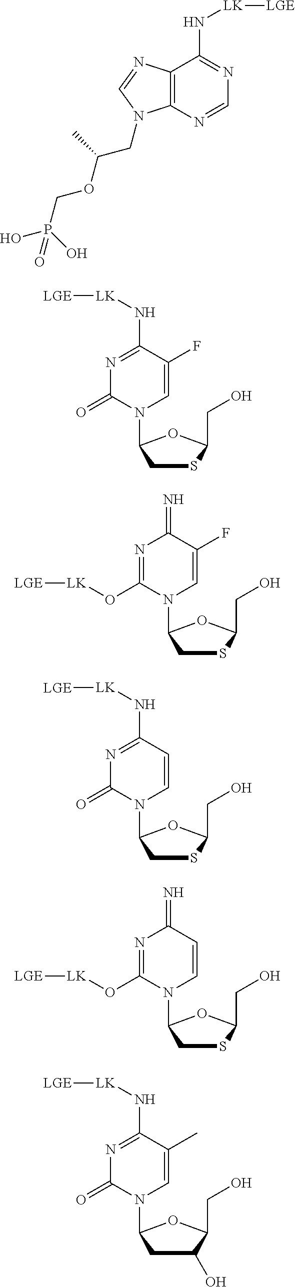 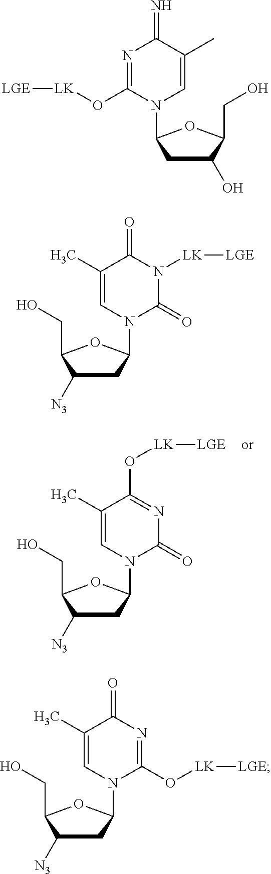
or preferably, the LK is linked to the cyclopentose unit of LGP, and further preferably, the linking position is shown in any of the following structures:

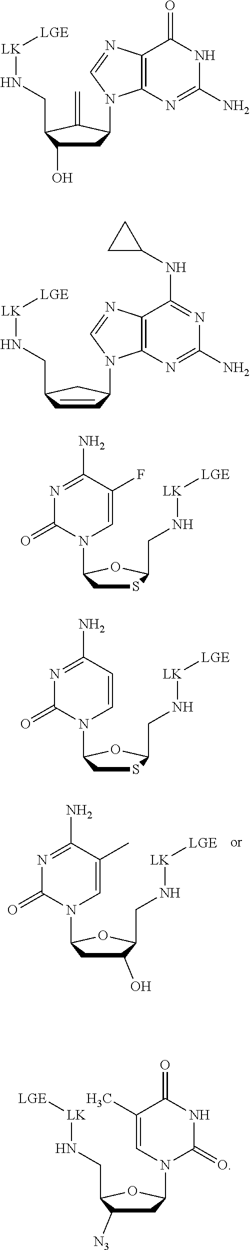

5. The bifunctional compound of claim 1, wherein the LGP-LK-LGE optionally has the structure shown below: 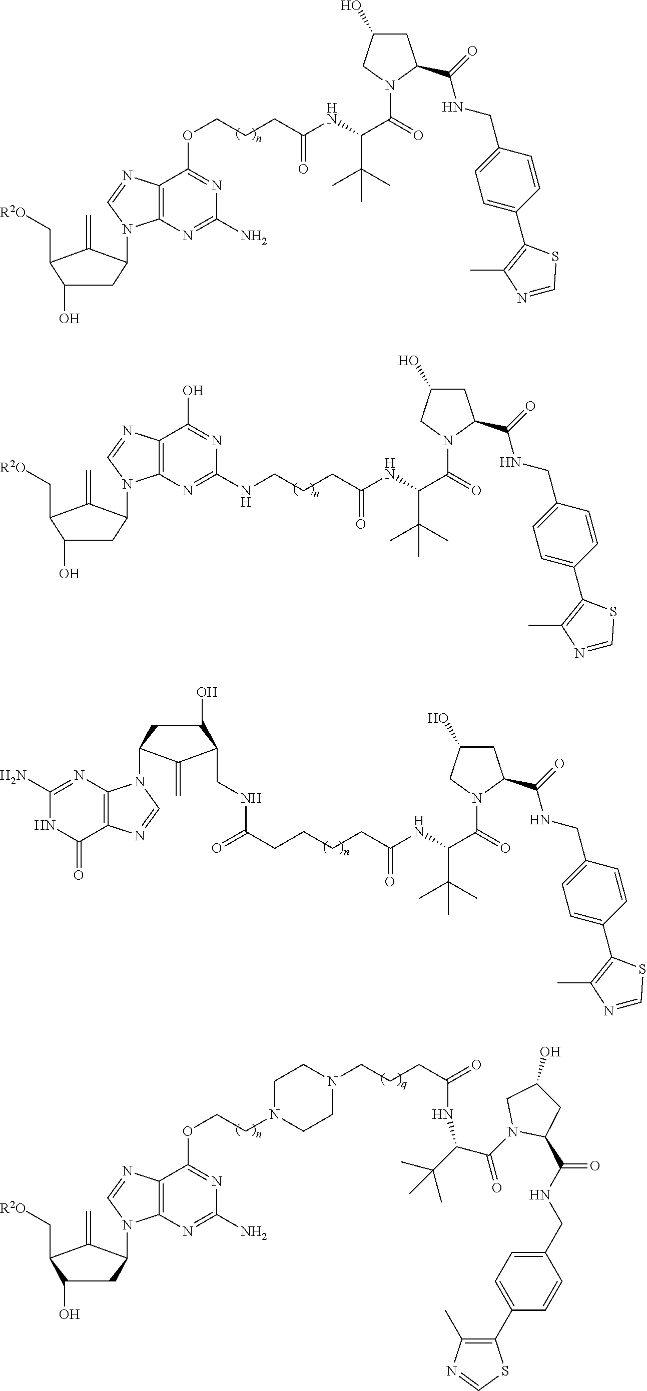 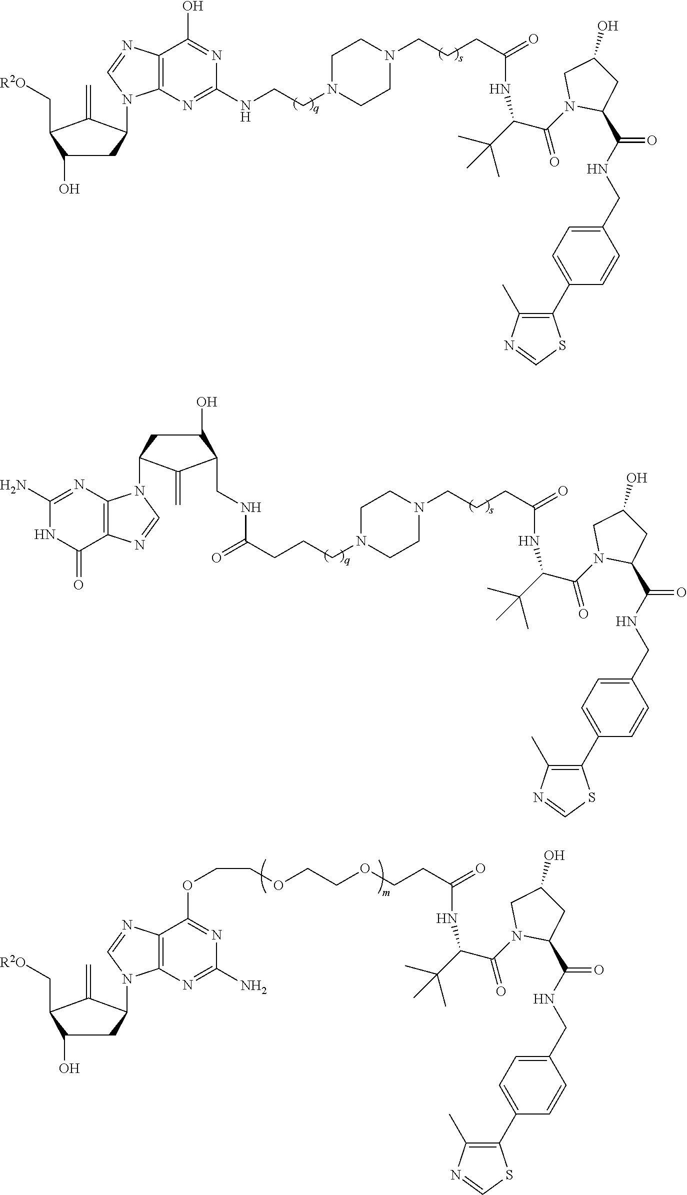 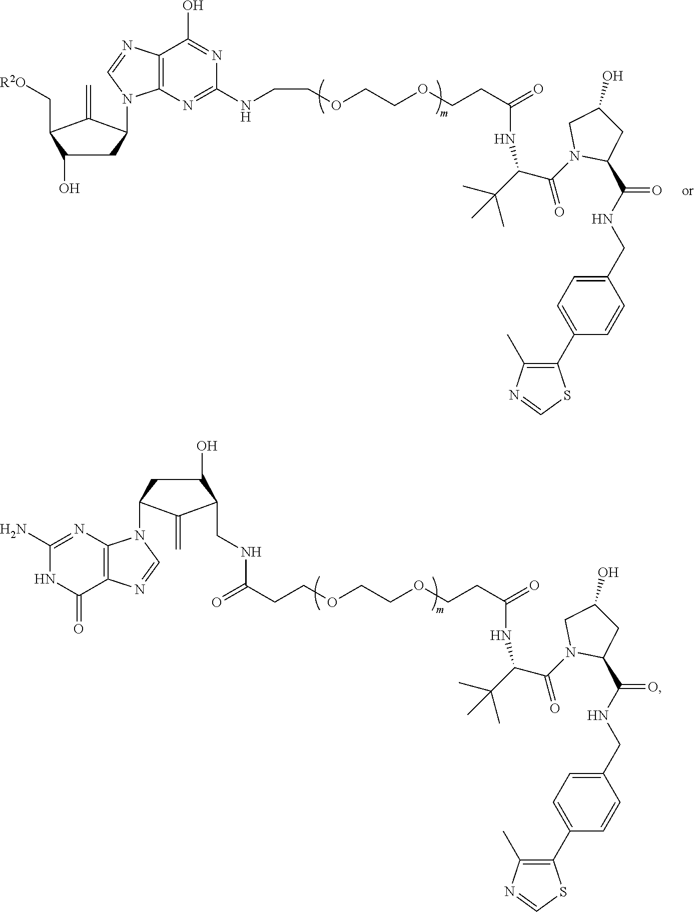
wherein, m is an integer of 0 to 5, n is an integer of 0 to 25, s is optionally an integer of 1 to 5, q is optionally an integer of 0 to 20, R² is optionally hydrogen, —P(O)(OH)₂, —P(O)OH—P(O)(OH)₂ or —P(O)(OH)—P(O)(OH)—P(O)(OH)₂; or 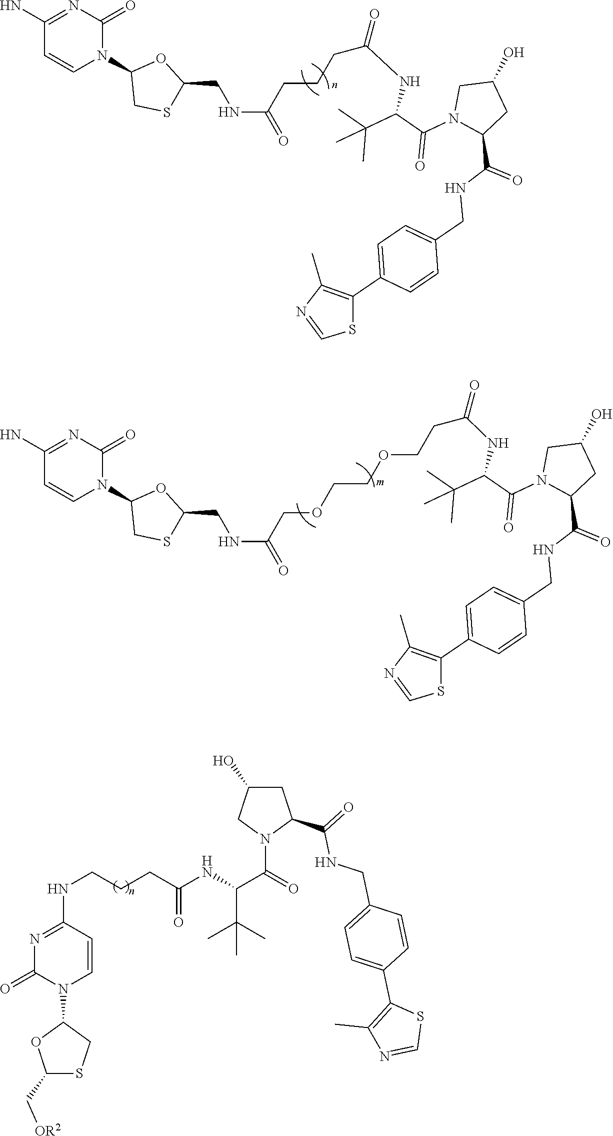 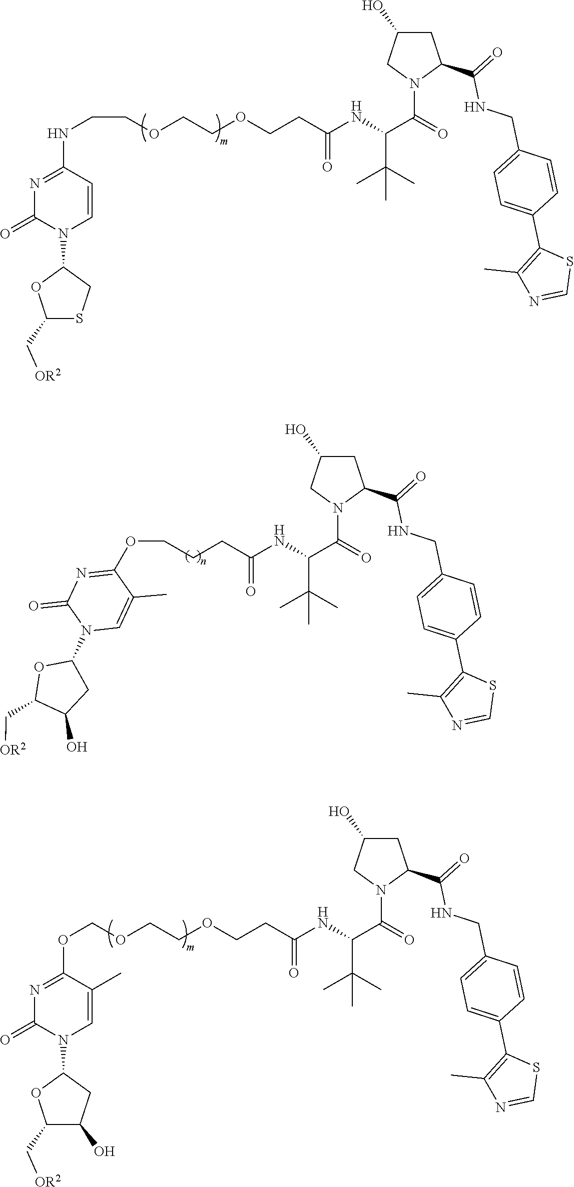 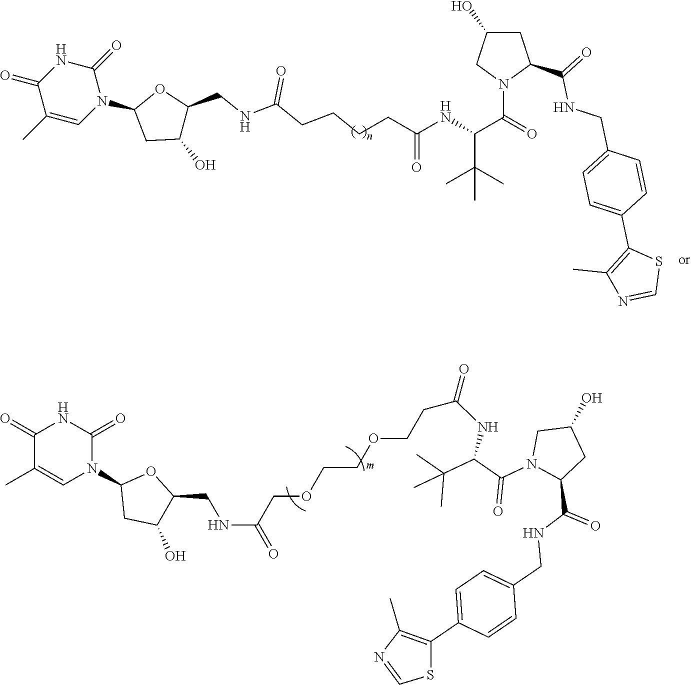
wherein, m is an integer of 0 to 5, n is an integer of 0 to 25, R² is optionally hydrogen, —P(O)(OH)₂, —P(O)OH—P(O)(OH)₂ or —P(O)(OH)—P(O)(OH)—P(O)(OH)₂; or 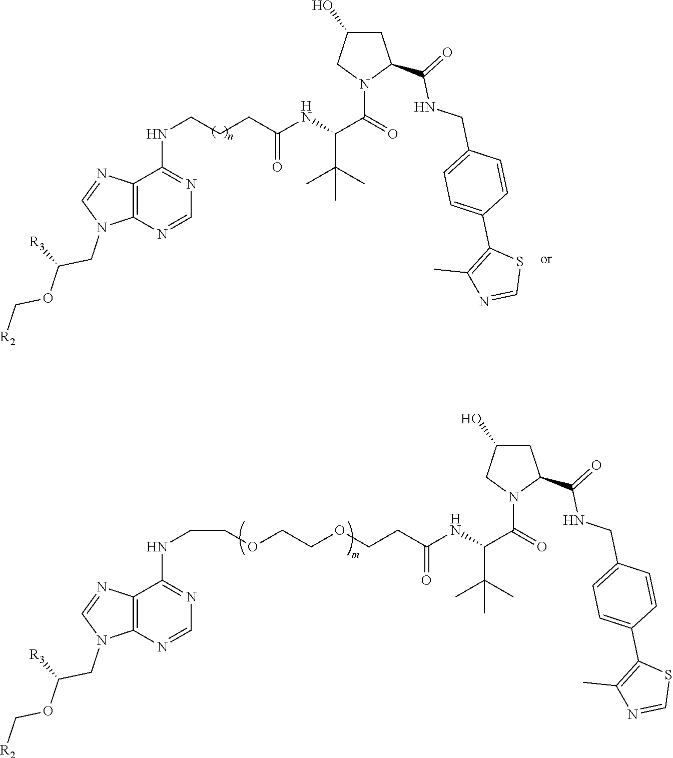
wherein, m is an integer of 0 to 5, n is an integer of 0 to 25, R² is optionally hydrogen, —P(O)(OH)₂, —P(O)OH—P(O)(OH)₂ or —P(O)(OH)—P(O)(OH)—P(O)(OH)₂, R³ is optionally hydrogen or methyl; or the bifunctional compound of claim 1, wherein, the LGP-LK-LG are preferably selected from the group consisting of:

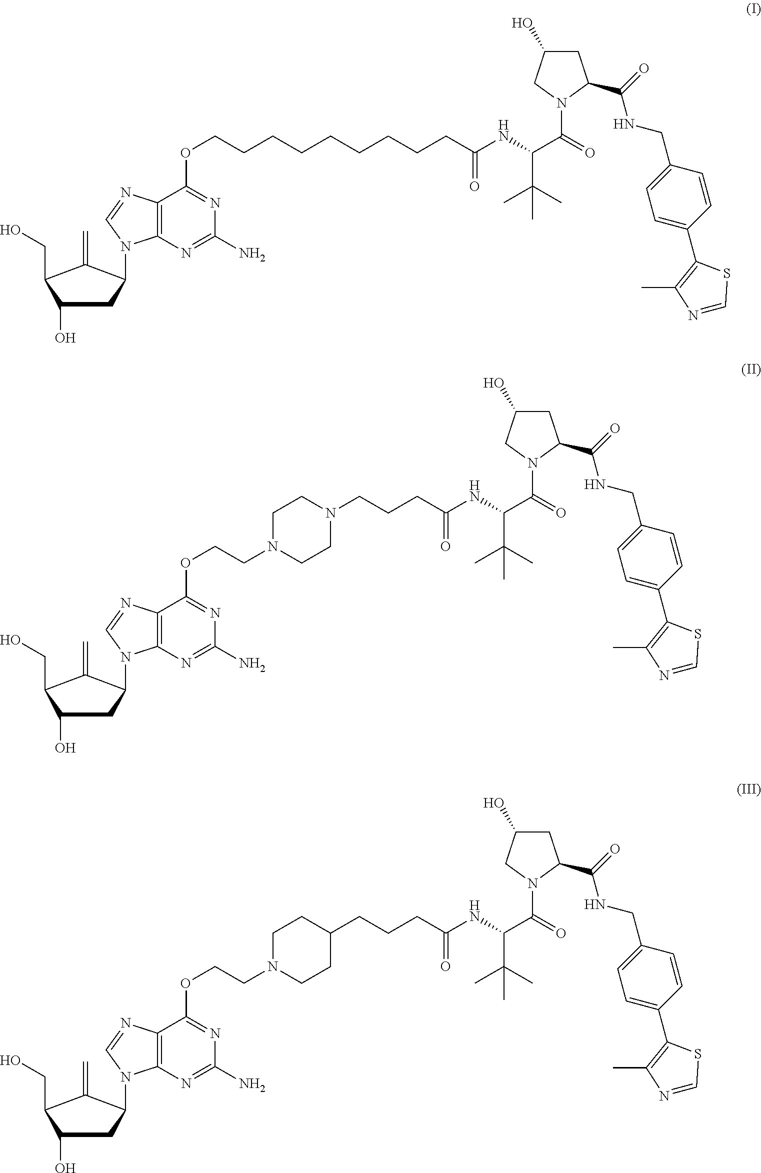 [SMILES: C=C1[C@H](CO)[C@H](O)C[C@@H]1n1cnc2c(OCCCCCCCCCC(=O)N[C@H](C(=O)N3C[C@H](O)C[C@H]3C(=O)NCc3ccc(-c4scnc4C)cc3)C(C)(C)C)nc(N)nc21 ; C=C1[C@H](CO)[C@H](O)C[C@@H]1n1cnc2c(OCCN3CCN(CCCC(=O)N[C@H](C(=O)N4C[C@H](O)C[C@H]4C(=O)NCc4ccc(-c5scnc5C)cc4)C(C)(C)C)CC3)nc(N)nc21 ; C=C1[C@H](CO)[C@H](O)C[C@@H]1n1cnc2c(OCCN3CCC(CCCC(=O)N[C@H](C(=O)N4C[C@H](O)C[C@H]4C(=O)NCc4ccc(-c5scnc5C)cc4)C(C)(C)C)CC3)nc(N)nc21]

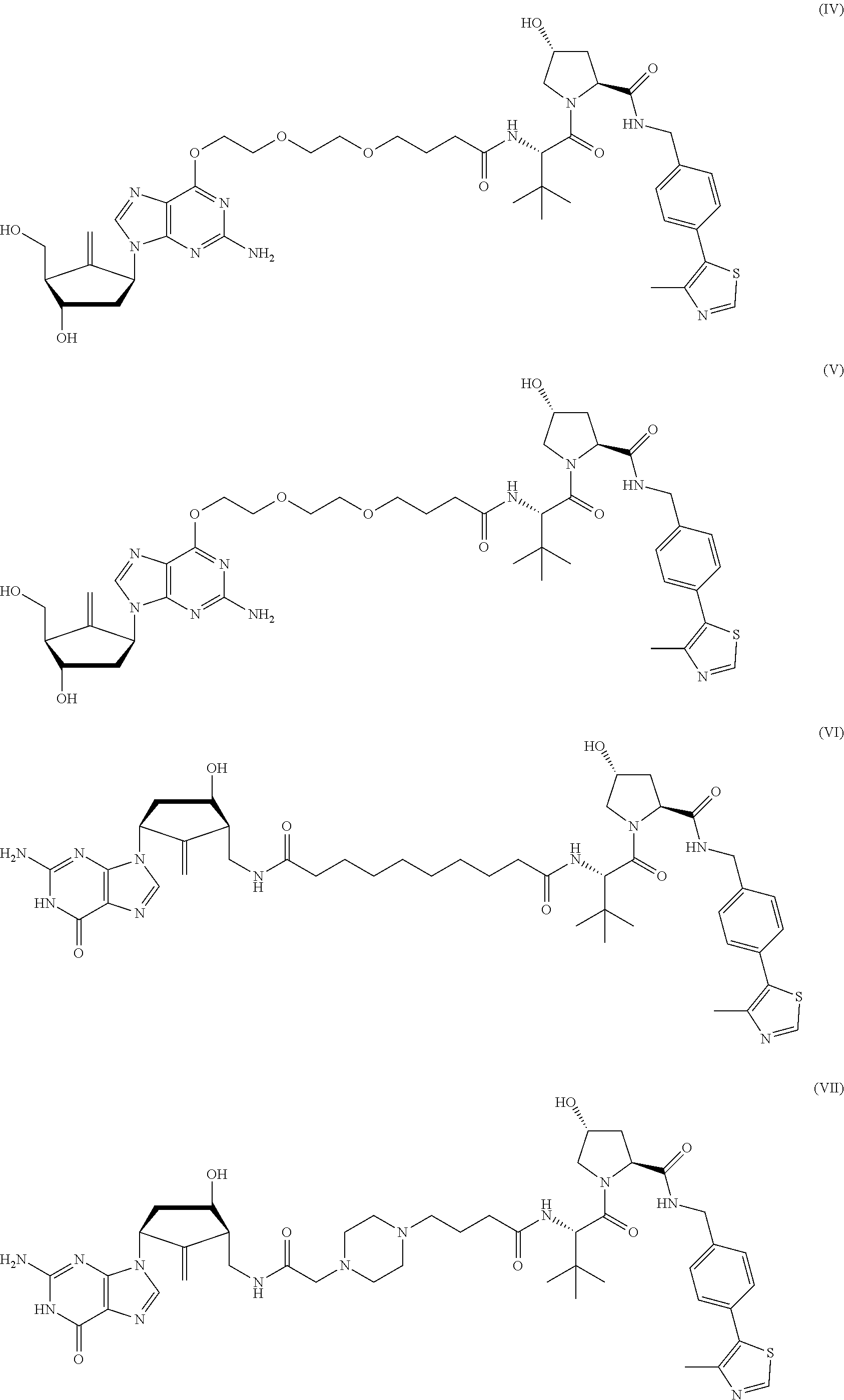

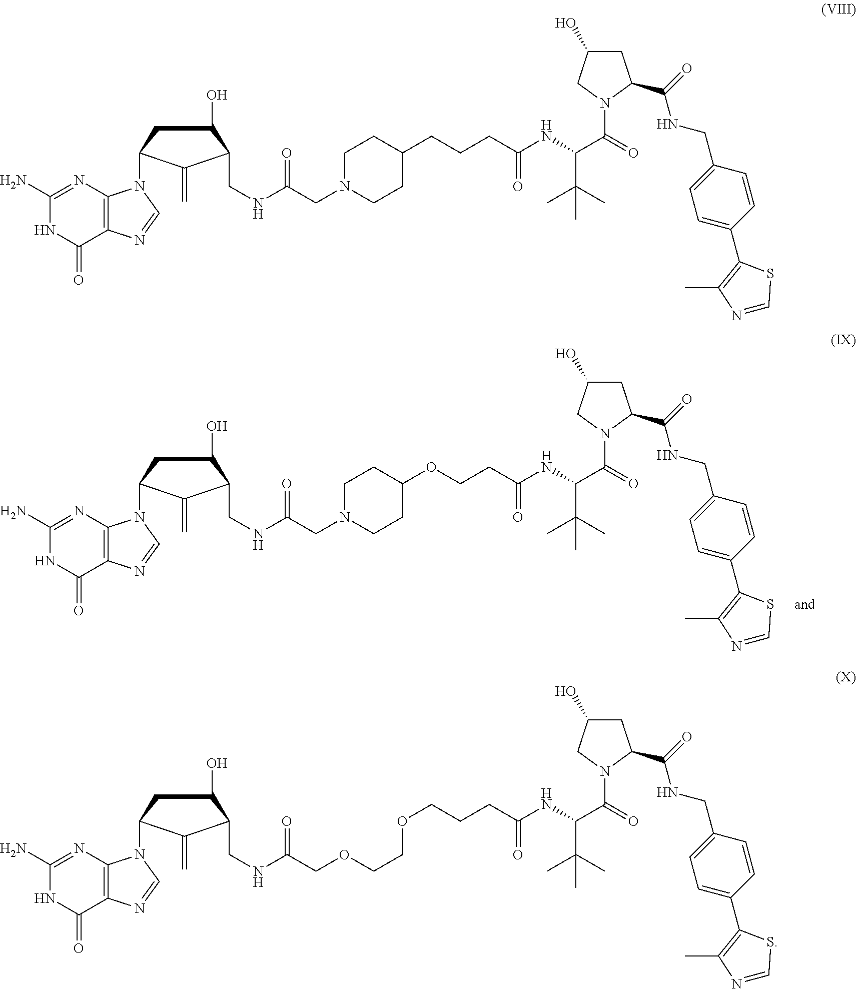 [SMILES: C=C1[C@H](CNC(=O)CN2CCC(CCCC(=O)N[C@H](C(=O)N3C[C@H](O)C[C@H]3C(=O)NCc3ccc(-c4scnc4C)cc3)C(C)(C)C)CC2)[C@H](O)C[C@@H]1n1cnc2c(=O)[nH]c(N)nc21 ; C=C1[C@H](CNC(=O)CN2CCC(OCCC(=O)N[C@H](C(=O)N3C[C@H](O)C[C@H]3C(=O)NCc3ccc(-c4scnc4C)cc3)C(C)(C)C)CC2)[C@H](O)C[C@@H]1n1cnc2c(=O)[nH]c(N)nc21 ; C=C1[C@H](CNC(=O)COCCOCCCC(=O)N[C@H](C(=O)N2C[C@H](O)C[C@H]2C(=O)NCc2ccc(-c3scnc3C)cc2)C(C)(C)C)[C@H](O)C[C@@H]1n1cnc2c(=O)[nH]c(N)nc21]

6. A pharmaceutical composition comprising the bifunctional compound of claim 1.

7. The pharmaceutical composition of claim 6, further comprises a pharmaceutically acceptable carrier, excipient, diluent, adjuvant, vehicle or a combination thereof.

8. A preparation method of the compound of claim 1, wherein, the compound (I) can be optionally prepared from the following two synthetic routes: 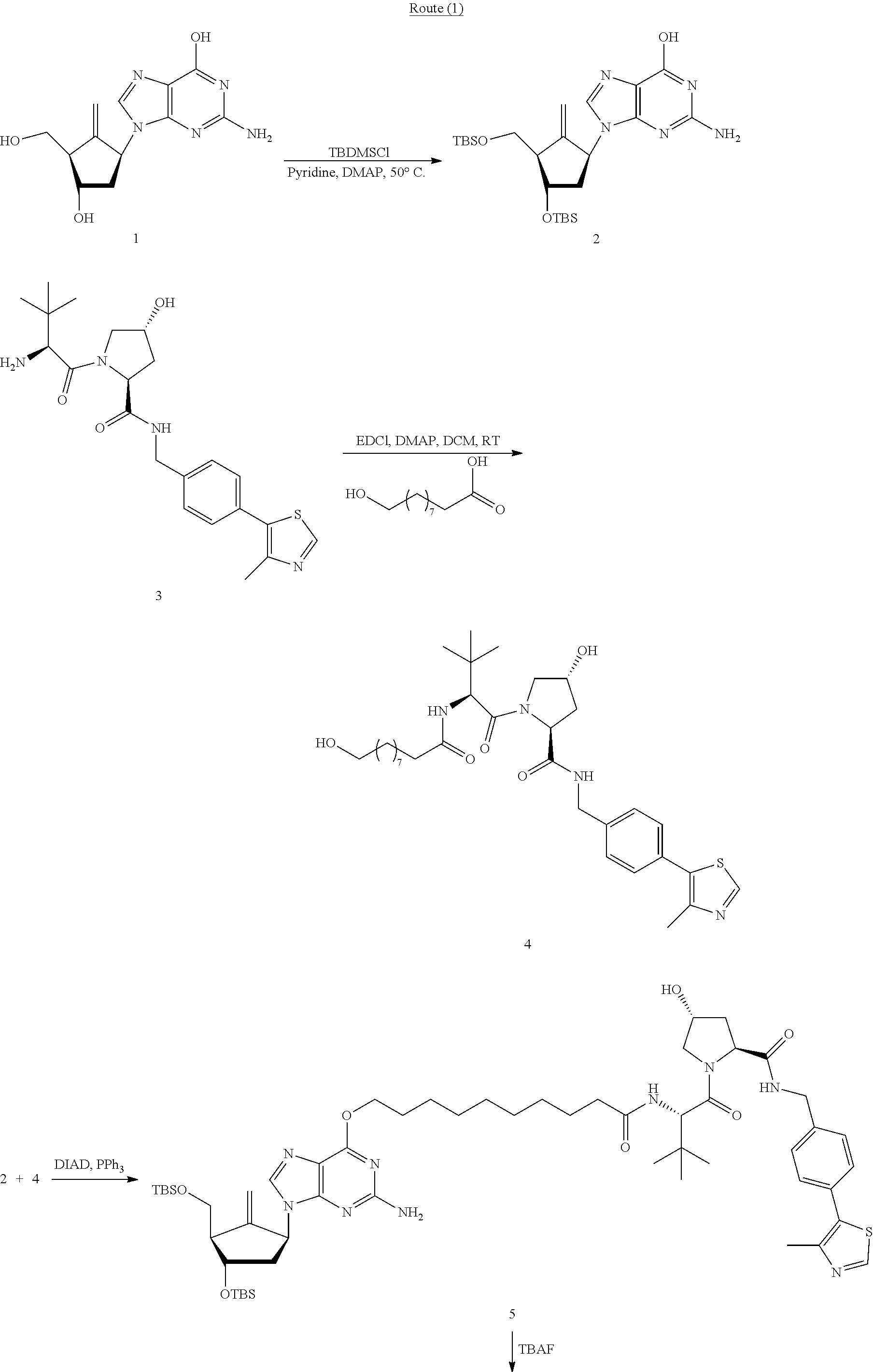 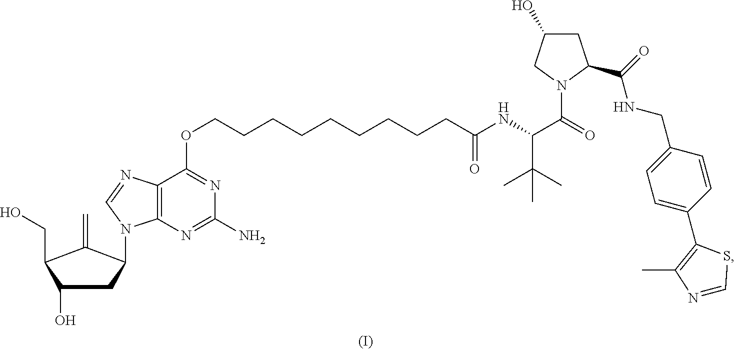 [SMILES: C=C1[C@H](CO)[C@H](O)C[C@@H]1n1cnc2c(OCCCCCCCCCC(=O)N[C@H](C(=O)N3C[C@H](O)C[C@H]3C(=O)NCc3ccc(-c4scnc4C)cc3)C(C)(C)C)nc(N)nc21 ; "(I)"] 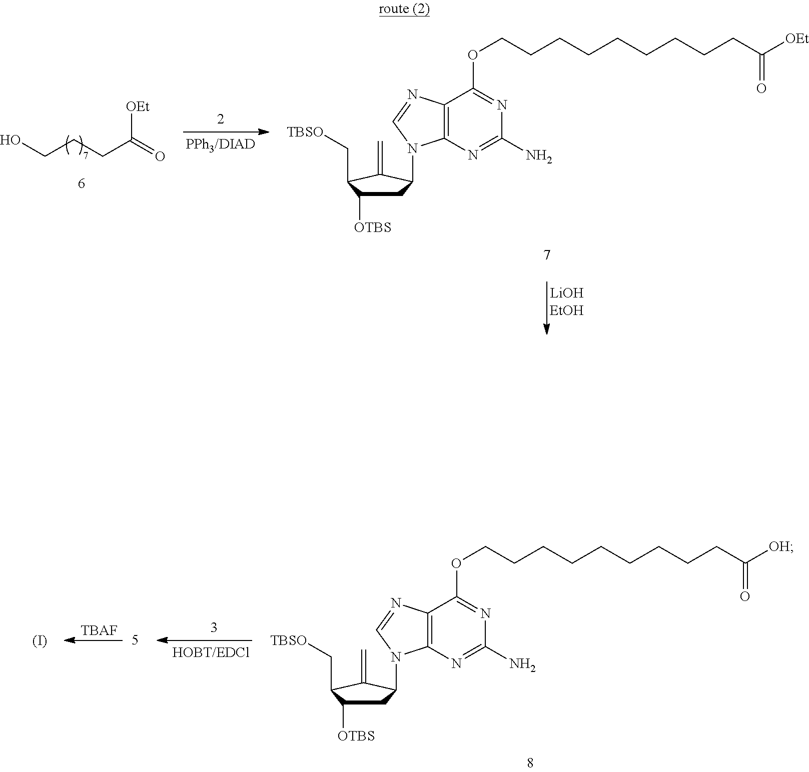
or, the compound (VI) can be prepared by the following synthetic route:

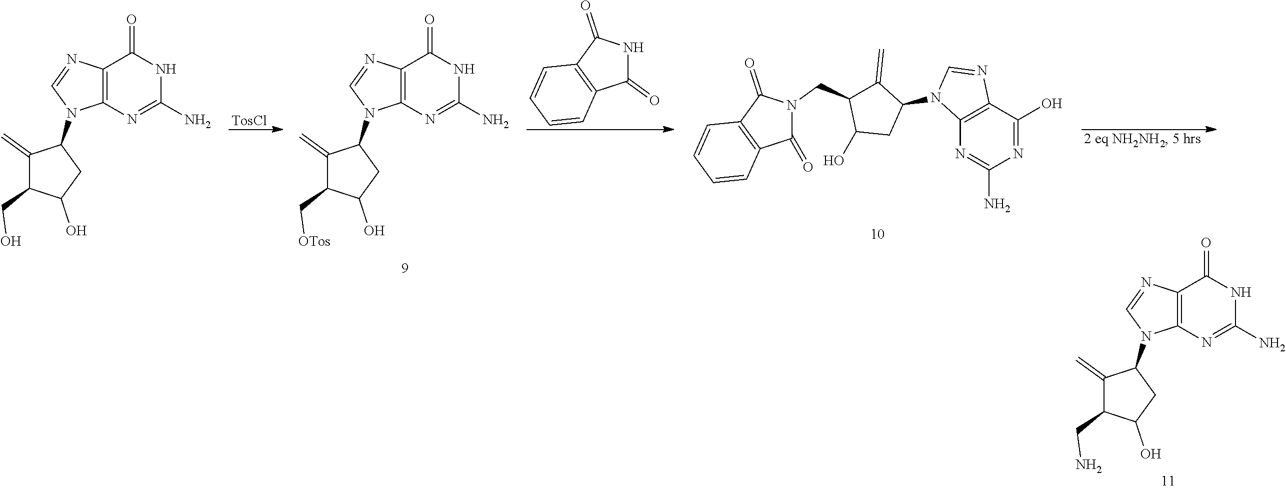

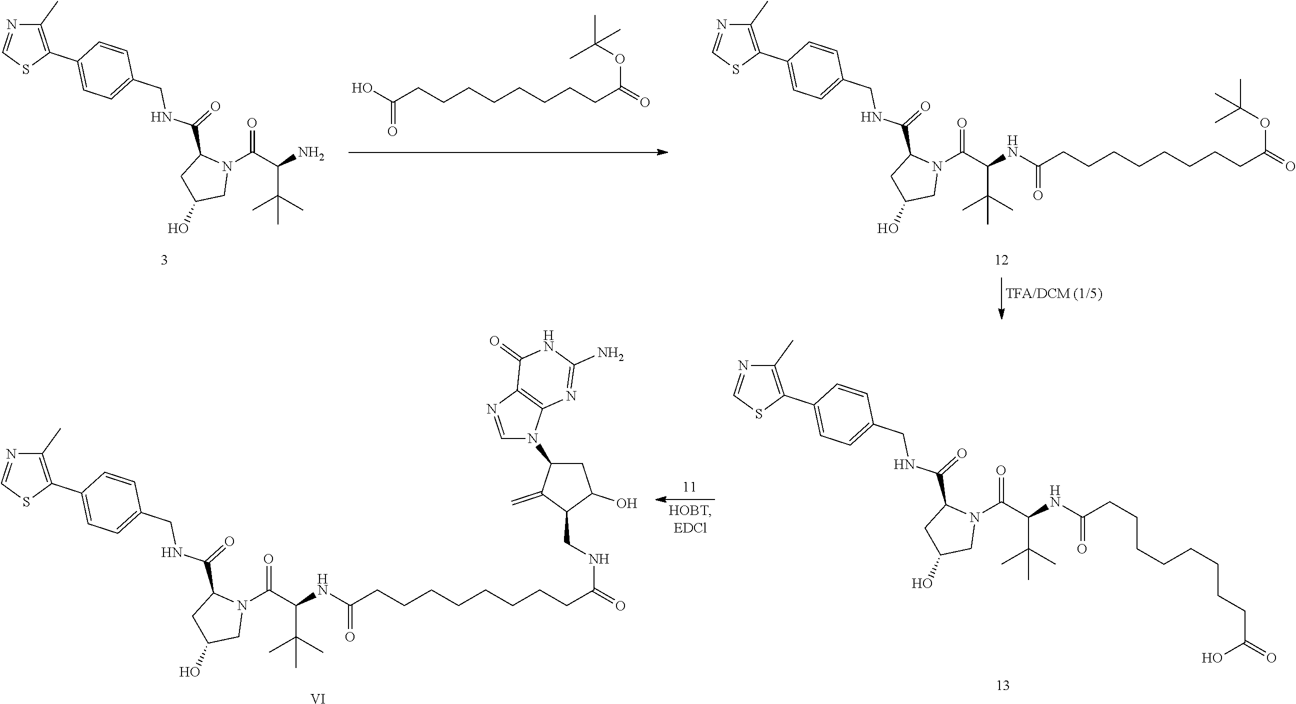

9. A method for degrading and inhibiting deoxyribonucleic acid (DNA) polymerase, wherein the method comprises using the bifunctional compound of claim 1, wherein the deoxyribonucleic acid (DNA) polymerase is preferably viral endogenous deoxyribonucleic acid (DNA) polymerase, more preferably hepatitis B virus endogenous deoxyribonucleic acid (DNA) polymerase, further preferably hepadnaviridae virus endogenous deoxyribonucleic acid (DNA) polymerases.

10. A method for treating, preventing or diagnosing various diseases related to DNA polymerase, wherein the method comprises using the bifunctional compound of claim 1, wherein the diseases include but are not limited to viral infectious diseases and secondary diseases thereof, preferably, the viral infectious diseases is infected by hepatitis B virus, human immunodeficiency virus, HCV, HDV, HEV, Ebola virus, SARS virus or COVID19; wherein, the secondary disease caused by the viral infection is liver cirrhosis, liver fibrosis, liver ascites or liver cancer.

11. A cell line Huh7-HBP that simultaneously expresses LgBiT and HiBiT and highly expresses HBP gene, wherein, the cell line Huh7-HBP has HBP gene, and is preferably prepared by the following method:
(1) inserting LgBiT tag nucleotide sequence into the lentiviral vector pCDH-CMV-EFla-Neo to obtain a recombinant plasmid pCDH-CMV-LgBiT-EFla-Neo, carrying out lentivirus package on the recombinant plasmid pCDH-CMV-LgBiT-EFla-Neo together with lentivirus helper plasmids pMD2.G and pSPAX2, and transducing the packaged lentivirus into Huh7 cell line to obtain a new cell line Huh7-LgBiT;
(2) inserting the sequence of the target gene HBP with a HiBiT tag at the N-terminus into the lentiviral vector pLVX-Puro vector to obtain a recombinant plasmid pLVX-HBP-Puro, carrying out lentivirus package on the recombinant plasmid pLVX-HBP-Puro together with the lentiviral helper plasmids pMD2.G and pSPAX2, and transducing the packaged lentivirus into Huh7-LgBiT cell line to obtain the cell line Huh7-HBP which expresses both LgBiT and HiBiT and highly expresses the HBP gene.

12. A method for determining the content of viral DNA polymerase in cells, wherein the method comprises using the cell line Huh7-HBP of claim 11.

13. A method for degrading and inhibiting deoxyribonucleic acid (DNA) polymerase, wherein the method comprises using the pharmaceutical composition of claim 6, wherein the deoxyribonucleic acid (DNA) polymerase is preferably viral endogenous deoxyribonucleic acid (DNA) polymerase, more preferably hepatitis B virus endogenous deoxyribonucleic acid (DNA) polymerase, further preferably hepadnaviridae virus endogenous deoxyribonucleic acid (DNA) polymerases.

14. A method for treating, preventing or diagnosing various diseases related to DNA polymerase, wherein the method comprises using the pharmaceutical composition of claim 6, wherein the diseases include but are not limited to viral infectious diseases and secondary diseases thereof, preferably, the viral infectious diseases is infected by hepatitis B virus, human immunodeficiency virus, HCV, HDV, HEV, Ebola virus, SARS virus or COVID19; wherein, the secondary disease caused by the viral infection is liver cirrhosis, liver fibrosis, liver ascites or liver cancer.
